(19) United States
(12) Patent Application Publication
(10) Pub. No.: US 2023/0081497 A1
(43) Pub. Date: Mar. 16, 2023

(54) ORAL CARE COMPOSITION COMPRISING CUTTLEFISH BONE POWDER

(71) Applicant: VISIONATUROLAB INC., Blainville (CA)

(72) Inventor: David HAUPTMAN, Blainville (CA)

(21) Appl. No.: 17/766,487

(22) PCT Filed: Oct. 2, 2020
(86) PCT No.: PCT/CA2020/051325

§ 371 Date: Apr. 4, 2022

Related U.S. Application Data
(60) Provisional application No. 62/981,173, filed on Feb. 25, 2020.
(60) Provisional application No. 62/910,820, filed on Oct. 4, 2019.

(51) Int. Cl. A61K 8/98; A61K 8/19; C01F 11/18; A61Q 11/00

(52) U.S. Cl. CPC A61K 8/987; A61K 8/19; C01F 11/18; A61Q 11/00; C01P 2004/51; C01P 2004/61

(57) ABSTRACT

The present document describes an oral care composition comprising a cuttlefish bone powder, comprising particles having more than 95% (w/w) calcium carbonate content, a specific surface area of at least 5 m²/g, a mechanical hardness about 4.75 to 6.87 GPa, and at least 20% of said particles of the powder have a particle size of from about 50 microns to about 70 microns and a mean of about 60 microns, and a suitable carrier, and uses of the composition for oral hygiene.

### BACKGROUND OF THE INVENTION

### Field of the Invention

The subject matter disclosed generally relates to oral care compositions and uses thereof, and more specifically, the subject matter disclosed relates to oral care compositions comprising cuttlefish bone powder and uses thereof.

### Description of Related Art

Bacterial plaque and calculus are major etiological factors in the initiation and progression of periodontal disease. Dental calculus is a mineralized plaque and because it is porous, it can absorb various toxic products and consequently serves as an ideal substrate for subgingival microbial colonization. Hence, calculus should be accurately detected and thoroughly removed for adequate periodontal therapy. Many techniques have been used to identify and remove calculus deposits that are present on the teeth surface. Maintenance of proper hygiene of the oral cavity is one of the most important measures to be undertaken to fight gingivitis. The selection of the proper toothpaste is the first step in the fight with oral disease. Brushing efficiently to remove dental plaque has always been a concern against tooth wear and gum recession. As established before, brushing alone has little wearing effect and therefore, loss of enamel as a result of brushing is mainly the result of abrasives used in toothpastes. On the other hand, most of the cleaning action while brushing is linked to the abrasive materials in the toothpaste and therefore, their presence is essential for cleaning. Toothpastes that are available today in the market have different kinds of abrasives in their formula, such as calcite, calcite and aragonite, silicon dioxide, brushite, gibbsite, etc.

Moreover, toothpastes are commonly produced to serve multiple purposes simultaneously thus, possess a complex chemical composition. Ideally, the formulation of a toothpaste should be balanced to maintain maximum cleaning benefit while minimizing the abrasive damage to the teeth structure. Therefore, excessively abrasive materials can abrade the tooth surface away, resulting in undesirable tooth wear and sensitivity. Many factors define the degree of abrasivity of a given compound, including its hydration level; the size, hardness, shape, and concentration of the particulate components; source; purity; as well as the method it has been treated physically and chemically. Also, toothpastes may act as vehicles for antimicrobial agents that may have a preventive/therapeutic role in periodontal disease. The complex composition of toothpastes implies that it is necessary to ensure that the active ingredients are not inactivated in the process of production or delivery. For instance, calcium carbonate binds to sodium fluoride rendering the latter ineffective as an anti-caries agent. Therefore, the composition of toothpastes is critical for their effectiveness on oral health maintenance and safety for the oral cavity.

Cuttlefish is a marine cephalopod mollusk with endoskeleton (called cuttlebone—CB) which is composed of calcium carbonate in aragonite phase. The cuttlebone composition is very similar to human bone, easily available and very cheap to extract and process. CB is the dried internal body part of squid cuttlefish that can be ground into a powder. The main chemical component of CB is 87.3%-91.75% calcium carbonate and chitin. In addition, CB also contains trace amounts of silicon, aluminum, titanium, manganese, barium, and copper. Being a rich source of calcium, the powdered cuttlebone in the mouth assists in calcium redeposition onto the teeth, a feature that saliva provides naturally. Due to its unique composition, the use of powdered CB in toothpastes could be one step forward to the production of more effective toothpaste that offer the dual function of cleaning the teeth and helps repair them in the microscopic level. The present description assesses the efficacy of a toothpaste containing cuttlefish bone powder in calculus removal compared to a commercial anti-calculus toothpaste that contains synthetic calcium carbonate (CaCO₃) as abrasive.

### SUMMARY OF THE INVENTION

It is against the above background that the present invention provides certain advantages and advancements over the prior art.

According to an embodiment, there is provided an oral care composition comprising:
a cuttlefish bone powder, comprising particles having more than 95% (w/w) calcium carbonate content, a specific surface area of at least 5 m²/g, a mechanical hardness about 4.75 to 7 GPa, and at least 20% of the particles of the powder have a particle size of from about 50 microns to about 70 microns and a mean of about 60 microns, and
a suitable carrier.

In the cuttlefish bone powder, at least 60% of the particles of the powder have a particle size of from about 10 microns to about 70 microns; and/or at least 55% of the particles of the powder have a particle size of from about 10 microns to about 60 microns, and/or at least 50% of the particles of the powder have a particle size of from about 10 microns to about 50 microns, and/or at least 40% of the particles of the powder have a particle size of from about 10 microns to about 45 microns, and/or at least 35% of the particles of the powder have a particle size of from about 10 microns to about 40 microns, and/or at least 30% of the particles of the powder have a particle size of from about 10 microns to about 35 microns, and/or at least 25% of the particles of the powder have a particle size of from about 10 microns to about 30 microns, and/or at least 20% of the particles of the powder have a particle size of from about 10 microns to about 26 microns, and/or at least 15% of the particles of the powder have a particle size of from about 10 microns to about 23 microns, and/or at least 12% of the particles of the powder have a particle size of from about 10 microns to about 20 microns, and/or at least 8% of the articles of the powder have a particle size of from about 10 microns to about 17 microns, and/or at least 6% of the particles of the powder have a particle size of from about 10 microns to about 15 microns, and/or at least 90% of the particles of the powder have a particle size of from about 10 microns to about 175 microns.

The calcium carbonate content may be from about 95% to about 99.9%.

The particles may have a specific surface area of from about 5 to about 8 or from about 5.2 to about 5.3.

The particles may have a mechanical hardness of about 4.75.

The particles may have a mechanical hardness of about 6.87.

The particles may have a zeta potential of about −11.4 to about −12.6 mV.

The particles may have a zeta potential of about −12.

The cuttlefish bone powder may be from about 0.100% to about 20% (w/w) of the composition.

The oral care composition may further comprise an abrasive.

The abrasive may be a colloidal calcium, a colloidal silica, a hydrated silica, a sodium bicarbonate (NaHCO₃), aluminum hydroxide (Al(OH)₃), calcium carbonate (CaCO₃), a calcium hydrogen phosphate (CaHPO₄.2H₂O), an anhydrous calcium hydrogen phosphate, a silica, a zeolites, and hydroxyapatite (Ca₅(PO₄)₃OH), or a combination thereof.

The abrasive may be from about 0.100% to about 0.325% (w/w) of the composition.

The colloidal silica may be from about 0.100% to about 0.275% (w/w).

The colloidal silica may be from about 0.02% to about 0.08% (w/w) of the composition.

The oral care composition may further comprise a thickening agent.

The thickening agent may be a natural gum obtained from seaweeds; a natural gum obtained from non-marine botanical resource, a natural gum produced by bacterial fermentation, a starch, a pectin, a carboxymethyl cellulose, a hydroxypropyl cellulose, a methyl cellulose, a gelatin or a combination thereof.

The natural gums obtained from seaweeds may be chosen from agar (E406), alginic acid (E400), Sodium alginate (E401), potassium alginate, ammonium alginate, calcium alginate, carrageenan (E407), or a combination thereof.

The natural gum obtained from non-marine botanical resource may be chosen from acacia gum, gum arabic (E414), gum ghatti, gum tragacanth (E413), karaya gum (E416), guar gum (E412), locust bean gum (E410), beta-glucan, chicle gum, dammar gum, Glucomannan (E425), mastic gum, psyllium seed husks, spruce gum, tara gum (E417), or a combination thereof.

The natural gum produced by bacterial fermentation may be chosen from gellan gum (E418), Xanthan gum (E415), or a combination thereof.

The thickening agent may be from about 2% to about 66% (w/w) of the composition.

The thickening agent may be about 2.2% (w/w) of the composition.

The oral care composition may further comprise a humectant.

The humectant may be propylene glycol, hexylene glycol, butylene glycol, glyceryl triacetate, neoagarobiose, a sugar polyol, a polymeric polyol, quillaia, lactic acid, urea, glycerin, aloe vera gel, MP Diol, an alpha hydroxy acid, and honey.

The sugar polyols may be chosen from glycerol, sorbitol, xylitol, maltitol, and a combination thereof.

The polymeric polyol may be polydextrose, polyethylene glycol, polypropylene glycol, poly(tetramethylene ether) glycol, and a combination thereof.

The alpha hydroxy acid may be lactic acid.

The humectant may be glycerin.

The humectant may be from about 2% to about 5% (w/w) of the composition.

The oral care composition may further comprise an emulsifier.

The emulsifier may be lecithin, a vegetal pulp powder, a sodium citrate and citric acid, or a combination thereof.

The vegetal pulp powder may be chosen from citrus pulp powder, baobab pulp powder, mango pulp powder, tomato pulp powder, pumpkin pulp powder, guava pulp powder, papaya pulp powder and beet pulp powder, or a combination thereof.

The sodium citrate may be trisodium citrate.

The emulsifier may be from about 4% to about 10% (w/w) of the composition.

The oral composition may further comprise a surfactant.

The surfactant may be chosen from sodium lauryl sulfate, ammonium lauryl sulfate, sodium N-lauryl sarcosinate, sodium lauryl sulfoacetate, or a combination thereof.

The surfactant may be from about 1% to about 3% (w/w) of the composition.

The oral composition may further comprise a pH regulator.

The pH regulator may be chosen from citric acid and its derivatives, phosphoric acid and its derivatives, trisodium phosphate, sodium citrate, lactic acid, bicarbonic acid, or a combination thereof.

The pH regulator may be from about 0.1% to about 0.25% (w/w) of the composition.

The oral composition may further comprise a preservative.

The preservative may be chosen from a sorbitan sesquioleate derivative, sodium benzoate, benzoic acid, a eucalyptus extract, or a combination thereof.

The preservative may be from about 0.2% to about 2% (w/w) of the composition.

The oral composition may further comprise a solvent.

The solvent may be chosen from water, ethanol, isopropanol, sorbitol and glycerin.

The solvent may be from about 60% to about 99% (w/w) of the composition.

The oral composition may further comprise an antimicrobial agent.

The antimicrobial agent may be chosen from a natural essential oil, an antimicrobial phenolic compound, or a combination thereof.

The natural essential oil may be chosen from oils of anise, lemon oil, orange oil, oregano, rosemary oil, wintergreen oil, thyme oil, lavender oil, clove oil, hops, tea tree oil, citronella oil, wheat oil, barley oil, lemongrass oil, cedar leaf oil, cedar wood oil, cinnamon oil, fleagrass oil, geranium oil, sandalwood oil, violet oil, cranberry oil, eucalyptus oil, vervain oil, peppermint oil, gum benzoin, basil oil, fennel oil, fir oil, balsam oil, menthol, ocmea origanum oil, Hydastis carradensis oil, Berberidaceae daceae oil, Ratanhiae and Curcuma longa oil, sesame oil, macadamia nut oil, evening primrose oil, Spanish sage oil, Spanish rosemary oil, coriander oil, thyme oil, pimento berries oil, rose oil, bergamot oil, rosewood oil, chamomile oil, sage oil, clary sage oil, cypress oil, sea fennel oil, frankincense oil, ginger oil, grapefruit oil, jasmine oil, juniper oil, lime oil, mandarin oil, marjoram oil, myrrh oil, neroli oil, patchouli oil, pepper oil, black pepper oil, petitgrain oil, pine oil, rose otto oil, spearmint oil, spikenard oil, vetiver oil, or ylang ylang.

The antimicrobial phenolic compound may be chosen from carvacrol, thymol, eugenol, eucalyptol, menthol, or a combination thereof.

The antimicrobial agent may be from about 0.01% to about 10% (w/w) of the composition.

According to another embodiment, there is provided use of an oral composition of the present invention for oral hygiene.

According to another embodiment, there is provided the use of an oral composition for removal of calculus, for prevention of calculus formation, or a combination thereof.

According to another embodiment, there is provided method of cleaning an oral cavity comprising applying the oral composition the present invention to an oral cavity.

According to another embodiment, there is provided a method of preventing formation of, or of removing calculus in an oral cavity comprising applying the oral composition of the present invention to an oral cavity.

According to another embodiment, there is provided a method of separating a ventral chamber and a dorsal shield of a cuttlefish bone, the method comprising the step of:
a) contacting a middle portion of said dorsal shield with a water jet produced from about 5 cm to about 10 cm from said dorsal shield,
said water jet being produced at a non-perpendicular angle relative to said middle portion of said dorsal shield,
said water jet having a pulverization angle of about 25 to 40 degrees, and a pressure of about 4800 kPa to about 7600 kPa,
   causing separation of said dorsal shield from said ventral portion.

The cuttlefish bone may be provided under a counter current orientation relative to said water jet.

The non-perpendicular angle may be from about 15 to about 75 degrees.

The cuttlefish bone may be provided at a speed of about 0.25 m/s to about 0.75 m/s, counter current to said water jet.

### BRIEF DESCRIPTION OF THE DRAWINGS

Further features and advantages of the present disclosure will become apparent from the following detailed description, taken in combination with the appended drawing, in which:

FIG. 1A illustrates an embedded enamel/dentin sample.

FIG. 1B illustrates embedded calculus sample.

FIG. 1C illustrates a mechanical brushing system.

FIG. 1D illustrates the embedded enamel/dentin sample and embedded calculus sample mounted on the mechanical brushing system.

FIG. 2A illustrates XRD patterns for synthetic CaCO₃, cuttlefish bone (CB) powder and treated cuttlefish bone (TCB) powder.

FIG. 2B illustrates FTIR spectra for synthetic CaCO₃, cuttlefish bone (CB) powder and treated cuttlefish bone (TCB) powder.

FIG. 3 illustrates SEM micrographs the as received cuttlefish bone; cuttlefish bone (CB) powder; treated cuttlefish bone (TCB) powder and synthetic CaCO₃.

FIG. 4A illustrates surface area measurements using BET.

FIG. 4B illustrates abrasion depth of an oral care composition according to the present invention vs. commercial toothpaste on enamel.

FIG. 4C illustrates abrasion depth of an oral care composition according to the present invention vs commercial toothpaste on dentin.

FIG. 4D illustrates abrasion depth of an oral care composition according to the present invention vs commercial toothpaste on Calculus.

FIG. 5A illustrates profile measurements taken across the brushed area vs part of the sample not exposed during brushing (used as the reference in calculations) for a composition according to the present invention. The deepest point in each profile was recorded.

FIG. 5B illustrates profile measurements taken across the brushed area vs part of the sample not exposed during brushing (used as the reference in calculations) for a commercial toothpaste. The deepest point in each profile was recorded.

FIG. 6 illustrates the surface morphology of the human tooth cross section after immersion in CaCO₃ (Top) and TCB (bottom). Occlusal area of enamel with typical prismatic structure of enamel rods showing detail enamel prism, dentin (smooth) showing parallel section of dentine and tubules and calculus rough topographical features.

FIG. 7 illustrates SEM micrographs and corresponding EDX mapping of human calculus investigated on its cross section after immersion in solution contains 10 mg CaCO₃ (Top) and TCB (bottom).

FIG. 8 illustrates the zeta potential of enamel, dentin, calculus, CB and TCB and synthetic CaCO₃ particles at concentration 0.01 mg/mL in ddH₂O at pH 7.5. Data collected from 3 measurements and a total of 50 runs per sample.

FIG. 9 shows a diagram of a clinical trial to compare performance of an oral care composition of the present invention with a commercial toothpaste (Crest® Complete Tartar Control Whitening Plus Scope).

FIG. 10 shows a diagram of a clinical trial to compare performance of an oral care composition of the present invention with a commercial toothpaste (Crest® Complete Tartar Control Whitening Plus Scope).

FIG. 11 shows teeth from clinical trial study participants treated with the oral care composition of the present invention (G—Group; 1 and 2) with a commercial toothpaste (C).

FIG. 12 illustrates the Volpe-Manhold Calculus Index before and after using the toothpastes. Scores are expressed as MI mean±S.E.M. *=significantly different from baseline score (before using the toothpastes); #=significantly different from Crest® toothpaste group. V=significantly different from time 6 months score (p<0.05). Crest® toothpaste=commercial product.

FIG. 13 illustrates the Shaw & Murray Stain Index before and after using the toothpastes. Scores are expressed as MI mean±S.E.M. *=significantly different from baseline score (before using the toothpastes); #=significantly different from Crest® toothpaste group. V=significantly different from time 6 months score (p<0.05). Crest® toothpaste=commercial product.

FIG. 14 illustrates the Modified Gingival Index before and after using the toothpastes. Scores are expressed as MI mean±S.E.M. *=significantly different from baseline score (before using the toothpastes); #=significantly different from Crest® toothpaste group. V=significantly different from time 6 months score (p<0.05). Crest® toothpaste=commercial product.

FIG. 15 illustrates Quigley-Hain Plaque Index before and after using the toothpastes. Scores are expressed as MI mean±S.E.M. *=significantly different from baseline score (before using the toothpastes); #=significantly different from Crest® toothpaste group. V=significantly different from time 6 months score (p<0.05). Crest® toothpaste=commercial product.

FIG. 16 illustrates the results of patient satisfaction survey at month 3, 6 and nine. Brackets indicate significant differences between groups at p<0.05. Statistical analysis was done with Student t-test.

FIG. 17 illustrates the result of survey Question 3 on toothpaste texture at different time points. Bracket indicates significant difference between timepoints. Statistical analysis was done with one-way ANOVA with post-hoc Bonferroni test.

FIG. 18 illustrates particle size distribution of TCB according to an embodiment of the present invention.

FIG. 19 illustrates FTIR spectra for cuttlefish bone (CB) powder and treated cuttlefish bone (TCB) powder. Top refers to the full spectra, while bottom focuses on values between 950 to 1275 cm⁻¹.

FIG. 20 illustrates the subtraction of the FTIR spectra for treated cuttlefish bone (TCB) powder and cuttlefish bone (CB) powder.

FIG. 21 Top illustrates XRD patterns cuttlefish bone (CB) powder and treated cuttlefish bone (TCB) powder, Bottom illustrates the subtraction of the XRD patterns treated cuttlefish bone (TCB) powder and cuttlefish bone (CB) powder.

FIG. 22 illustrates FTIR spectra for Calcium carbonate (CaCO₃), cuttlefish bone (CB) powder and treated cuttlefish bone (TCB) powder. Bottom shows the spun down samples.

FIG. 23 illustrates FTIR spectra for Calcium carbonate (CaCO₃), cuttlefish bone (CB) powder and treated cuttlefish bone (TCB) powder after incubation in supersaturated solution of calcium phosphate.

FIG. 24 illustrates FTIR spectra for chitin and treated chitin powder after incubation in supersaturated solution of calcium phosphate. Top refers to the full spectra, while bottom the spun down samples.

FIG. 25A illustrates dental calculus samples before treatment with TCB (SEM).

FIG. 25B illustrates dental calculus samples after treatment with TCB (SEM).

FIG. 25C illustrates dental calculus samples before treatment with TCB (SEM).

FIG. 25D illustrates dental calculus samples after treatment with TCB (SEM).

FIG. 26 illustrates XRD patterns of calculus before or after exposure to cuttlefish bone (TCB) powder.

FIG. 27 illustrates a profilometry analysis of dental calculus after exposure to TCB slurry.

FIG. 28 illustrates the increase in the trends before and after treatment from the profilometry analysis shown in FIG. 27.

FIG. 29 illustrates XRD patterns of cuttlefish bone (TCB) powder after exposure to calculus.

FIG. 30 illustrates XRD patterns of cuttlefish bone (TCB) powder after exposure to calculus.

FIG. 31 illustrates XRD patterns of cuttlefish bone (TCB) powder after exposure to calculus.

FIG. 32 illustrates XRD patterns of cuttlefish bone (TCB) powder after exposure to calculus.

FIG. 33 illustrates an EDX elemental analysis of dental calculus before and after exposure to TCB slurry

FIG. 34 illustrates FTIR spectra for TCB upon exposure to dental calculus or distilled water.

FIG. 35 illustrates FTIR spectra for TCB upon exposure to dental calculus or distilled water.

FIG. 36 illustrates FTIR spectra for TCB upon exposure to dental calculus or distilled water.

FIG. 37 illustrates EDX elemental analysis of TCB before and after exposure to dental calculus.

FIG. 38 illustrates a possible reaction between dental calculus and TCB based on the results presented herein.

FIG. 39 illustrates a GA-XRD patterns of calculus after exposure to treated cuttlefish bone (TCB), 60-61 μm TCB and no treatment.

FIG. 40 Top illustrates the subtraction of the XRD patterns of treated cuttlefish bone (TCB) 60-61 μm powder and no treatment calculus, and Bottom illustrates the subtraction of the XRD patterns treated cuttlefish bone (TCB) powder and no treatment calculus.

FIG. 41 illustrates ATR-IR spectra of (Top) treated cuttlefish bone (TCB) 60-61 μm and (Bottom) treated cuttlefish bone (TCB) powders before and after reaction in a super-saturated solution of calcium phosphate.

FIG. 42 illustrates FTIR spectra of treated cuttlefish bone (TCB) 60-61 μm and (TCB) powders (Top) before and (Bottom) after reaction in a super-saturated solution of calcium phosphate.

FIG. 43 illustrates the spun down samples of treated cuttlefish bone (TCB) and treated cuttlefish bone (TCB) 60-61 μm.

FIG. 44 illustrates the dorsal shield side of a cuttlefish bone.

FIG. 45 illustrates the ventral chamber (or lamellar matrix) of a cuttlefish bone.

FIG. 46 illustrates a frontal view of a cuttlefish bone (dorsal shield on the bottom).

FIG. 47 illustrates a damaged cuttlebone piece showing the dorsal shield on top and the ventral chamber (lamellar matrix) underneath.

FIG. 48 illustrates a flowchart of the different processing steps leading to the preparation of TCB according to the present invention.

FIG. 49 is a schematic representation of cuttlebones (10) being contacted with water jet (20), when under movement counter-currently to the water jet.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

### DETAILED DESCRIPTION OF THE INVENTION

All publications, patents and patent applications cited herein are hereby expressly incorporated by reference for all purposes.

Before describing the present invention in detail, a number of terms will be defined. As used herein, the singular forms “a”, “an”, and “the” include plural referents unless the context clearly dictates otherwise.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Dental calculus is mineralized plaque and because it is porous, it can absorb various toxic chemicals, food debris and bacteria that can damage the periodontal tissues. Hence, calculus removal is critical for maintaining adequate periodontal health. Therefore, there has been substantial interest in the development and implementation of approaches that will ease the calculus removal process.

Therefore, according to an embodiment, there is disclosed an oral care composition which contains a natural bio-descaling powder of cuttlefish bone (CB).

In embodiments there are disclosed an oral care compositions. The compositions of the present invention are oral care compositions containing as an ingredient cuttlefish bone powder obtained from ground bones. Cuttlefish are marine animals of the order Sepiida. They belong to the class Cephalopoda, which also includes squid, octopuses, and nautiluses. “Cuttle” is a reference to their unique internal shell, the cuttlebone. Despite their name, cuttlefish are not fish but mollusks.

A cuttlefish possesses an internal structure called the cuttlebone, which is porous and is made of aragonite. Aragonite is a carbonate mineral, one of the two common, naturally occurring, crystal forms of calcium carbonate, CaCO₃ (the other form being the mineral calcite). It is formed by biological and physical processes, including precipitation from marine and freshwater environments.

Aragonite's crystal lattice differs from that of calcite, resulting in a different crystal shape, an orthorhombic system with acicular crystals. Repeated twinning results in pseudo-hexagonal forms. Aragonite may be columnar or fibrous, occasionally in branching stalactitic forms called flos-ferri (“flowers of iron”) from their association with the ores at the Carinthian iron mines.

Aragonite forms naturally in almost all mollusk shells, and as the calcareous endoskeleton of warm- and cold-water corals (Scleractinia). Several serpulids have aragonitic tubes. Because the mineral deposition in mollusk shells is strongly biologically controlled, some crystal forms are distinctively different from those of inorganic aragonite. In some mollusks, the entire shell is aragonite; in others, aragonite forms only discrete parts of a bimineralic shell (aragonite plus calcite). Aragonite also forms in the ocean and in caves as inorganic precipitates called marine cements and speleothems, respectively. The nacreous layer of the aragonite fossil shells of some extinct ammonites forms an iridescent material called ammolite. Ammolite is primarily aragonite with impurities that make it iridescent and valuable as a gemstone.

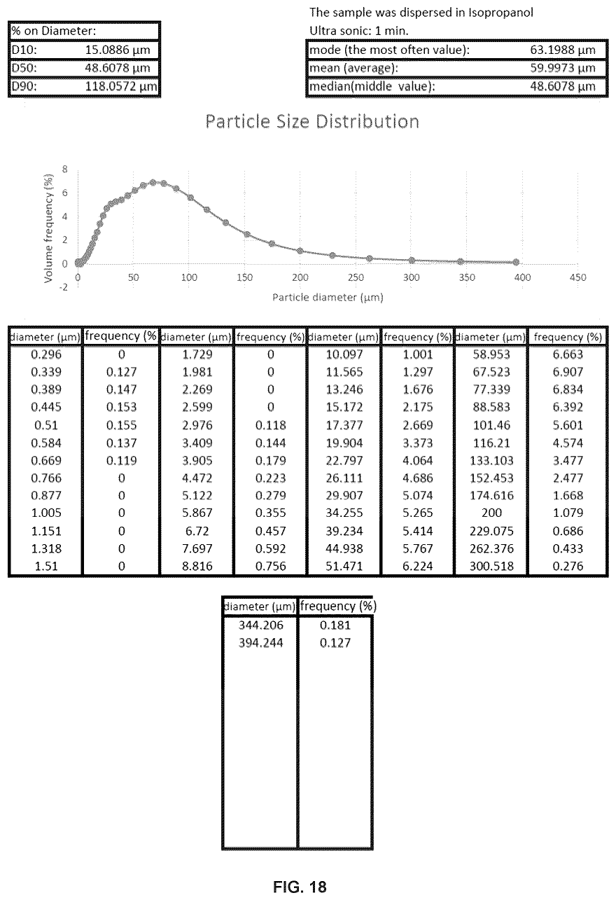

The particles of the cuttlefish bone powder used in the present invention may particles according to the particles described in FIG. 18 which shows that the TCB comprises a very small amount of (less than 1%) of very fine particles having diameters of less than 0.67 μm, and less than 4% of total particles having diameters equal to 8.8 μm or less. Particles of about 10 μm to about 20 μm represent about 12% of total particles, particles of above 20 μm up to about 30 μm represent about 13.8% of total particles, particles of above 30 μm up to about 40 μm represent about 10.7% of total particles, particles of above 40 μm up to about 45 μm represent about 5.8% of total particles, particles of above 45 μm up to about 60 μm represent about 12.9% of total particles, particles of above 60 μm up to about 68 μm represent about 6.9% of total particles, particles of above 68 μm up to about 77 μm represent about 6.8% of total particles, particles of above 77 μm up to about 89 μm represent about 6.4% of total particles, particles of above 89 μm up to about 101 μm represent about 5.6% of total particles, particles of above 101 μm up to about 116 μm represent about 4.6% of total particles, particles of above 116 μm up to about 133 μm represent about 3.5% of total particles, particles of above 133 μm up to about 152 μm represent about 2.5% of total particles, particles of above 152 μm up to about 175 μm represent about 1.7% of total particles, and particles of above 175 μm up to about 400 μm represent about 4.6% of total particles. Therefore, according to embodiments, the cuttlefish bone powder may be comprising particles having more than 95% (w/w) calcium carbonate content, a specific surface area of at least 5, a mechanical hardness about 4.75 to 7 GPa, and at least 20% of the particles of the powder have a particle size of from about 50 microns to about 70 microns and a mean of about 60 microns.

In embodiments, at least 60% of the particles of the powder have a particle size of from about 10 microns to about 70 microns, and/or at least 55% of the particles of the powder have a particle size of from about 10 microns to about 60 microns, and/or at least 50% of the particles of the powder have a particle size of from about 10 microns to about 50 microns, and/or at least 40% of the particles of the powder have a particle size of from about 10 microns to about 45 microns, and/or at least 35% of the particles of the powder have a particle size of from about 10 microns to about 40 microns, and/or at least 30% of the particles of the powder have a particle size of from about 10 microns to about 35 microns, and/or at least 25% of the particles of the powder have a particle size of from about 10 microns to about 30 microns, and/or at least 20% of the particles of the powder have a particle size of from about 10 microns to about 26 microns, and/or at least 15% of the particles of the powder have a particle size of from about 10 microns to about 23 microns, and/or at least 12% of the particles of the powder have a particle size of from about 10 microns to about 20 microns, and/or at least 8% of the particles of the powder have a particle size of from about 10 microns to about 17 microns, and/or at least 6% of the particles of the powder have a particle size of from about 10 microns to about 15 microns, and/or at least 90% of the particles of the powder have a particle size of from about 10 microns to about 175 microns.

The favored abrasion ration value is between 0 and 88 in accordance to the DESAUTELS and LABRECHE 1999 scale. The abrasiveness scale of DESAUTELS and LABRECHE varies as follows for toothpaste: 1) bit abrasive: 0% to 88%; 2) abrasive to medium abrasive: 88% to 100% and 3) very abrasive: >100%.

Specific surface area (SSA) is a property of solids defined as the total surface area of a material per unit of mass. The particles of treated cuttlefish bone powder of the present invention may have a specific surface area of at least 5 m²/g, or at least 5.1 m²/g, or at least 5.2 m²/g, or at least 5.3 m²/g, or at least 5.4 m²/g, or at least 5.5 m²/g, or at least 5.6 m²/g, or at least 5.7 m²/g, or at least 5.8 m²/g, or at least 5.9 m²/g, or at least 6.0 m²/g, or at least 6.1 m²/g, or at least 6.2 m²/g, or at least 6.3 m²/g, or at least 6.4 m²/g, or at least 6.5 m²/g, or at least 6.6 m²/g, or at least 6.7 m²/g, or at least 6.8 m²/g, or at least 6.9 m²/g, or at least 7.0 m²/g, or at least 7.1 m²/g, or at least 7.2 m²/g, or at least 7.3 m²/g, or at least 7.4 m²/g, or at least 7.5 m²/g, or at least 7.6 m²/g, or at least 7.7 m²/g, or at least 7.8 m²/g, or at least 7.9 m²/g, or at least 8.0 m²/g, for example from about 5 to about 5.356 m²/g, or from about 5.290±0.0660 m²/g, or from about 5 m²/g to about 5.1 m²/g, or from about 5 m²/g to about 5.2 m²/g, or from about 5 m²/g to about 5.3 m²/g, or from about 5 m²/g to about 5.4 m²/g, or from about 5 m²/g to about 5.5 m²/g, or from about 5 m²/g to about 5.6 m²/g, or from about 5 m²/g to about 5.7 m²/g, or from about 5 m²/g to about 5.8 m²/g, or from about 5 m²/g to about 5.9 m²/g, or from about 5 m²/g to about 6.0 m²/g, or from about 5 m²/g to about 6.1 m²/g, or from about 5 m²/g to about 6.2 m²/g, or from about 5 m²/g to about 6.3 m²/g, or from about 5 m²/g to about 6.4 m²/g, or from about 5 m²/g to about 6.5 m²/g, or from about 5 m²/g to about 7.0 m²/g, or from about 5 m²/g to about 7.5 m²/g, or from about 5 m²/g to about 8.0 m²/g, or from about 5.1 m²/g to about 5.2 m²/g, or from about 5.1 m²/g to about 5.3 m²/g, or from about 5.1 m²/g to about 5.4 m²/g, or from about 5.1 m²/g to about 5.5 m²/g, or from about 5.1 m²/g to about 5.6 m²/g, or from about 5.1 m²/g to about 5.7 m²/g, or from about 5.1 m²/g to about 5.8 m²/g, or from about 5.1 m²/g to about 5.9 m²/g, or from about 5.1 m²/g to about 6.0 m²/g, or from about 5.1 m²/g to about 6.1 m²/g, or from about 5.1 m²/g to about 6.2 m²/g, or from about 5.1 m²/g to about 6.3 m²/g, or from about 5.1 m²/g to about 6.4 m²/g, or from about 5.1 m²/g to about 6.5 m²/g, or from about 5.1 m²/g to about 7.0 m²/g, or from about 5.1 m²/g to about 7.5 m²/g, or from about 5.1 m²/g to about 8.0 m²/g, from about 5.2 m²/g to about 5.3 m²/g, or from about 5.2 m²/g to about 5.4 m²/g, or from about 5.2 m²/g to about 5.5 m²/g, or from about 5.2 m²/g to about 5.6 m²/g, or from about 5.2 m²/g to about 5.7 m²/g, or from about 5.2 m²/g to about 5.8 m²/g, or from about 5.2 m²/g to about 5.9 m²/g, or from about 5.2 m²/g to about 6.0 m²/g, or from about 5.2 m²/g to about 6.1 m²/g, or from about 5.2 m²/g to about 6.2 m²/g, or from about 5.2 m²/g to about 6.3 m²/g, or from about 5.2 m²/g to about 6.4 m²/g, or from about 5.2 m²/g to about 6.5 m²/g, or from about 5.2 m²/g to about 7.0 m²/g, or from about 5.2 m²/g to about 7.5 m²/g, or from about 5.2 m²/g to about 8.0 m²/g, from about 5.3 m²/g to about 5.4 m²/g, or from about 5.3 m²/g to about 5.5 m²/g, or from about 5.3 m²/g to about 5.6 m²/g, or from about 5.3 m²/g to about 5.7 m²/g, or from about 5.3 m²/g to about 5.8 m²/g, or from about 5.3 m²/g to about 5.9 m²/g, or from about 5.3 m²/g to about 6.0 m²/g, or from about 5.3 m²/g to about 6.1 m²/g, or from about 5.3 m²/g to about 6.2 m²/g, or from about 5.3 m²/g to about 6.3 m²/g, or from about 5.3 m²/g to about 6.4 m²/g, or from about 5.3 m²/g to about 6.5 m²/g, or from about 5.3 m²/g to about 7.0 m²/g, or from about 5.3 m²/g to about 7.5 m²/g, or from about 5.3 m²/g to about 8.0 m²/g, or from about 5.4 m²/g to about 5.5 m²/g, or from about 5.4 m²/g to about 5.6 m²/g, or from about 5.4 m²/g to about 5.7 m²/g, or from about 5.4 m²/g to about 5.8 m²/g, or from about 5.4 m²/g to about 5.9 m²/g, or from about 5.4 m²/g to about 6.0 m²/g, or from about 5.4 m²/g to about 6.1 m²/g, or from about 5.4 m²/g to about 6.2 m²/g, or from about 5.4 m²/g to about 6.3 m²/g, or from about 5.4 m²/g to about 6.4 m²/g, or from about 5.4 m²/g to about 6.5 m²/g, or from about 5.4 m²/g to about 7.0 m²/g, or from about 5.4 m²/g to about 7.5 m²/g, or from about 5.4 m²/g to about 8.0 m²/g, or from about 5.5 m²/g to about 5.6 m²/g, or from about 5.5 m²/g to about 5.7 m²/g, or from about 5.5 m²/g to about 5.8 m²/g, or from about 5.5 m²/g to about 5.9 m²/g, or from about 5.5 m²/g to about 6.0 m²/g, or from about 5.5 m²/g to about 6.1 m²/g, or from about 5.5 m²/g to about 6.2 m²/g, or from about 5.5 m²/g to about 6.3 m²/g, or from about 5.5 m²/g to about 6.4 m²/g, or from about 5.5 m²/g to about 6.5 m²/g, or from about 5.5 m²/g to about 7.0 m²/g, or from about 5.5 m²/g to about 7.5 m²/g, or from about 5.5 m²/g to about 8.0 m²/g, or from about 5.6 m²/g to about 5.7 m²/g, or from about 5.6 m²/g to about 5.8 m²/g, or from about 5.6 m²/g to about 5.9 m²/g, or from about 5.6 m²/g to about 6.0 m²/g, or from about 5.6 m²/g to about 6.1 m²/g, or from about 5.6 m²/g to about 6.2 m²/g, or from about 5.6 m²/g to about 6.3 m²/g, or from about 5.6 m²/g to about 6.4 m²/g, or from about 5.6 m²/g to about 6.5 m²/g, or from about 5.6 m²/g to about 7.0 m²/g, or from about 5.6 m²/g to about 7.5 m²/g, or from about 5.6 m²/g to about 8.0 m²/g, or from about 5.7 m²/g to about 5.8 m²/g, or from about 5.7 m²/g to about 5.9 m²/g, or from about 5.7 m²/g to about 6.0 m²/g, or from about 5.7 m²/g to about 6.1 m²/g, or from about 5.7 m²/g to about 6.2 m²/g, or from about 5.7 m²/g to about 6.3 m²/g, or from about 5.7 m²/g to about 6.4 m²/g, or from about 5.7 m²/g to about 6.5 m²/g, or from about 5.7 m²/g to about 7.0 m²/g, or from about 5.7 m²/g to about 7.5 m²/g, or from about 5.7 m²/g to about 8.0 m²/g, or from about 5.8 m²/g to about 5.9 m²/g, or from about 5.8 m²/g to about 6.0 m²/g, or from about 5.8 m²/g to about 6.1 m²/g, or from about 5.8 m²/g to about 6.2 m²/g, or from about 5.8 m²/g to about 6.3 m²/g, or from about 5.8 m²/g to about 6.4 m²/g, or from about 5.8 m²/g to about 6.5 m²/g, or from about 5.8 m²/g to about 7.0 m²/g, or from about 5.3 m²/g to about 7.5 m²/g, or from about 5.3 m²/g to about 8.0 m²/g, or from about 5.9 m²/g to about 6.0 m²/g, or from about 5.9 m²/g to about 6.1 m²/g, or from about 5.9 m²/g to about 6.2 m²/g, or from about 5.9 m²/g to about 6.3 m²/g, or from about 5.9 m²/g to about 6.4 m²/g, or from about 5.9 m²/g to about 6.5 m²/g, or from about 5.9 m²/g to about 7.0 m²/g, or from about 5.9 m²/g to about 7.5 m²/g, or from about 5.9 m²/g to about 8.0 m²/g, or from about 6.0 m²/g to about 6.1 m²/g, or from about 6.0 m²/g to about 6.2 m²/g, or from about 6.0 m²/g to about 6.3 m²/g, or from about 6.0 m²/g to about 6.4 m²/g, or from about 6.0 m²/g to about 6.5 m²/g, or from about 6.0 m²/g to about 7.0 m²/g, or from about 6.0 m²/g to about 7.5 m²/g, or from about 6.0 m²/g to about 8.0 m²/g, or from about 6.1 m²/g to about 6.2 m²/g, or from about 6.1 m²/g to about 6.3 m²/g, or from about 6.1 m²/g to about 6.4 m²/g, or from about 6.1 m²/g to about 6.5 m²/g, or from about 6.1 m²/g to about 7.0 m²/g, or from about 6.1 m²/g to about 7.5 m²/g, or from about 6.1 m²/g to about 8.0 m²/g, or from about 6.2 m²/g to about 6.3 m²/g, or from about 6.2 m²/g to about 6.4 m²/g, or from about 6.2 m²/g to about 6.5 m²/g, or from about 6.2 m²/g to about 7.0 m²/g, or from about 6.2 m²/g to about 7.5 m²/g, or from about 6.2 m²/g to about 8.0 m²/g, or from about 6.3 m²/g to about 6.4 m²/g, or from about 6.3 m²/g to about 6.5 m²/g, or from about 6.3 m²/g to about 7.0 m²/g, or from about 6.3 m²/g to about 7.5 m²/g, or from about 6.3 m²/g to about 8.0 m²/g, or from about 6.4 m²/g to about 6.5 m²/g, or from about 6.4 m²/g to about 7.0 m²/g, or from about 6.4 m²/g to about 7.5 m²/g, or from about 6.4 m²/g to about 8.0 m²/g, or from about 6.5 m²/g to about 7.0 m²/g, or from about 6.5 m²/g to about 7.5 m²/g, or from about 6.5 m²/g to about 8.0 m²/g, or from about 7.0 m²/g to about 7.5 m²/g, or from about 7.0 m²/g to about 8.0 m²/g, or from about 7.5 m²/g to about 8.0 m²/g.

In embodiments, the particles of the treated cuttlefish bone powder used in the oral care composition of the present invention may have mechanical hardness about 4.75 to about 7.0 GPa, or from about 4.75 to about 6.87 GPa, or from about 4.75 to about 6.8 GPa, or from about 4.75 to about 6.5 GPa, or from about 4.75 to about 6.0 GPa, or from about 4.75 to about 5.5 GPa, or from about 4.75 to about 5.0 GPa, or from about 5.0 to 7.0 G about Pa, or from about 5.0 to 6.87 GPa, or from about 5.0 to about 6.8 GPa, or from about 5.0 to about 6.5 GPa, or from about 5.0 to about 6.0 GPa, or from about 5.0 to about 5.5 GPa, or from about 5.5 to about 7.0 GPa, or from about 5.5 to about 6.87 GPa, or from about 5.5 to about 6.8 GPa, or from about 5.5 to about 6.5 GPa, or from about 5.5 to about 6.0 GPa, or from about 6.0 to about 7.0 GPa, or from about 6.0 to about 6.87 GPa, or from about 6.0 to about 6.8 GPa, or from about 6.0 to about 6.5 GPa, or from about 6.5 to about 7.0 GPa, or from about 6.5 to about 6.87 GPa, or from about 6.5 to about 6.8 GPa.

The particles have been treated with a mild demineralization treatment, for example in ammonium chloride or ammonium acetate, at pH from about 4.5 to about 5.5, or from about 4.5 to about 5.4, or from about 4.5 to about 5.3, or from about 4.5 to about 5.2, or from about 4.5 to about 5.1, or from about 4.5 to about 5.0, or from about 4.5 to about 4.9, or from about 4.5 to about 4.8, or from about 4.5 to about 4.7, or from about 4.5 to about 4.6, or from about 4.6 to about 5.5, or from about 4.6 to about 5.4, or from about 4.6 to about 5.3, or from about 4.6 to about 5.2, or from about 4.6 to about 5.1, or from about 4.6 to about 5.0, or from about 4.6 to about 4.9, or from about 4.6 to about 4.8, or from about 4.6 to about 4.7, or from about 4.7 to about 5.5, or from about 4.7 to about 5.4, or from about 4.7 to about 5.3, or from about 4.7 to about 5.2, or from about 4.7 to about 5.1, or from about 4.7 to about 5.0, or from about 4.7 to about 4.9, or from about 4.7 to about 4.8, or from about 4.8 to about 5.5 or from about 4.8 to about 5.4, or from about 4.8 to about 5.3, or from about 4.8 to about 5.2, or from about 4.8 to about 5.1, or from about 4.8 to about 5.0, or from about 4.8 to about 4.9, or from about 4.9 to about 5.5, or from about 4.9 to about 5.4, or from about 4.9 to about 5.3, or from about 4.9 to about 5.2, or from about 4.9 to about 5.1, or from about 4.9 to about 5.0, or from about 5.0 to about 5.5, or from about 5.0 to about 5.4, or from about 5.0 to about 5.3, or from about 5.0 to about 5.2, or from about 5.0 to about 5.1, or from about 5.1 to about 5.5, or from about 5.1 to about 5.4, or from about 5.1 to about 5.3, or from about 5.1 to about 5.2, or from about 5.2 to about 5.5, or from about 5.2 to about 5.4, or from about 5.2 to about 5.3, or from about 5.3 to about 5.5, or from about 5.3 to about 5.4, or from about 5.4 to about 5.5, and preferably pH about 4.75, 4.76, 4.77, 4.78, 4.79, 4.80, 4.81, 4.82, 4.83, 4.84, 4.85, 4.86, 4.87, 4.88, 4.89, 4.90, and most preferably 4.86 in order to solubilize unwanted magnesium, ammonia, iron and zinc compounds present in the bone material, and increase the calcium carbonate content of the powder of the present invention. Indeed, the bone powder used in the present invention comprises a high content in calcium; containing at least 95% calcium carbonate, with reduced amounts of magnesium, zinc, iron and ammonia containing derivatives. According to embodiments, the calcium carbonate of the cuttlefish bone powder particles may be at least 95%, at least 95.5%, at least 96%, at least 96.5%, at least 97%, at least 97.5%, at least 98%, at least 98.5%, at least 99%, or from about 95% to 99% (w/w), or from about 95% to 98.5% (w/w), or from about 95% to about 98%, or from about 95% to 97.5% (w/w), or from about 95% to about 97%, or from about 95% to 96.5% (w/w), or from about 95% to about 96%, or from about 95% to 95.5% (w/w), or from about 95.5% to 99% (w/w), or from about 95.5% to 98.5% (w/w), or from about 95.5% to about 98%, or from about 95.5% to 97.5% (w/w), or from about 95.5% to about 97%, or from about 95.5% to 96.5% (w/w), or from about 95.5% to about 96%, or from about 96% to 99% (w/w), or from about 96% to 98.5% (w/w), or from about 96% to about 98%, or from about 96% to 97.5% (w/w), or from about 96% to about 97%, or from about 96% to 96.5% (w/w), or from about 96.5% to 99% (w/w), or from about 96.5% to 98.5% (w/w), or from about 96.5% to about 98%, or from about 96.5% to 97.5% (w/w), or from about 96.5% to about 97%, or from about 97% to 99% (w/w), or from about 97% to 98.5% (w/w), or from about 97% to about 98%, or from about 97% to 97.5% (w/w), or from about 97.5% to 99% (w/w), or from about 97.5% to 98.5% (w/w), or from about 97.5% to about 98%, or from about 98% to 99% (w/w), or from about 98% to 98.5% (w/w), or from about 98.5% to 99% (w/w).

The compositions of the present invention are suitable for eliminating tartar, or at least help reduce the presence of dental tartar. Furthermore, the compositions of the present invention do not include any source of fluoride ions. Fluoride containing products have a normally low pH around 4.5, which favors its action, but contributes to demineralization of the enamel and root. The cuttlefish bone power component of the present invention contains a high amount of calcium, which may on the contrary contribute to remineralization of the enamel and root.

The compositions of the present invention may contribute to teeth whitening without further addition of hydrogen peroxide, nor hexametaphosphate, tripolyphosphate, or enzymes which are currently part of whitening toothpaste compositions.

According to embodiments of the present invention, the cuttlefish bone powder described above may represent from about 3% to about 25% (w/w), or from about 3% to about 24%, or from about 3% to about 23%, or from about 3% to about 22%, or from about 3% to about 21%, or from about 3% to about 20%, or from about 3% to about 19%, or from about 3% to about 18%, or from about 3% to about 17%, or from about 3% to about 16%, or from about 3% to about 15%, or from about 3% to about 14%, or from about 3% to about 13%, or from about 3% to about 12%, or from about 3% to about 11%, or from about 3% to about 10%, or from about 3% to about 9%, or from about 3% to about 8%, or from about 3% to about 7%, or from about 3% to about 6%, or from about 3% to about 5%, or from about 3% to about 4%, or from about 4% to about 25% (w/w), or from about 4% to about 24%, or from about 4% to about 23%, or from about 4% to about 22%, or from about 4% to about 21%, or from about 4% to about 20%, or from about 4% to about 19%, or from about 4% to about 18%, or from about 4% to about 17%, or from about 4% to about 16%, or from about 4% to about 15%, or from about 4% to about 14%, or from about 4% to about 13%, or from about 4% to about 12%, or from about 4% to about 11%, or from about 4% to about 10%, or from about 4% to about 9%, or from about 4% to about 8%, or from about 4% to about 7%, or from about 4% to about 6%, or from about 4% to about 5%, or from about 5% to about 25% (w/w), or from about 5% to about 24%, or from about 5% to about 23%, or from about 5% to about 22%, or from about 5% to about 21%, or from about 5% to about 20%, or from about 5% to about 19%, or from about 5% to about 18%, or from about 5% to about 17%, or from about 5% to about 16%, or from about 5% to about 15%, or from about 5% to about 14%, or from about 5% to about 13%, or from about 5% to about 12%, or from about 5% to about 11%, or from about 5% to about 10%, or from about 5% to about 9%, or from about 5% to about 8%, or from about 5% to about 7%, or from about 5% to about 6%, or from about 6% to about 25% (w/w), or from about 6% to about 24%, or from about 6% to about 23%, or from about 6% to about 22%, or from about 6% to about 21%, or from about 6% to about 20%, or from about 6% to about 19%, or from about 6% to about 18%, or from about 6% to about 17%, or from about 6% to about 16%, or from about 6% to about 15%, or from about 6% to about 14%, or from about 6% to about 13%, or from about 6% to about 12%, or from about 6% to about 11%, or from about 6% to about 10%, or from about 6% to about 9%, or from about 6% to about 8%, or from about 6% to about 7%, or from about 7% to about 25% (w/w), or from about 7% to about 24%, or from about 7% to about 23%, or from about 7% to about 22%, or from about 7% to about 21%, or from about 7% to about 20%, or from about 7% to about 19%, or from about 7% to about 18%, or from about 7% to about 17%, or from about 7% to about 16%, or from about 7% to about 15%, or from about 7% to about 14%, or from about 7% to about 13%, or from about 7% to about 12%, or from about 7% to about 11%, or from about 7% to about 10%, or from about 7% to about 9%, or from about 7% to about 8%, or from about 8% to about 25% (w/w), or from about 8% to about 24%, or from about 8% to about 23%, or from about 8% to about 22%, or from about 8% to about 21%, or from about 8% to about 20%, or from about 8% to about 19%, or from about 8% to about 18%, or from about 8% to about 17%, or from about 8% to about 16%, or from about 8% to about 15%, or from about 8% to about 14%, or from about 8% to about 13%, or from about 8% to about 12%, or from about 8% to about 11%, or from about 8% to about 10%, or from about 8% to about 9%, or from about 9% to about 25% (w/w), or from about 9% to about 24%, or from about 9% to about 23%, or from about 9% to about 22%, or from about 9% to about 21%, or from about 9% to about 20%, or from about 9% to about 19%, or from about 9% to about 18%, or from about 9% to about 17%, or from about 9% to about 16%, or from about 9% to about 15%, or from about 9% to about 14%, or from about 9% to about 13%, or from about 9% to about 12%, or from about 9% to about 11%, or from about 9% to about 10%, or from about 10% to about 25% (w/w), or from about 10% to about 24%, or from about 10% to about 23%, or from about 10% to about 22%, or from about 10% to about 21%, or from about 10% to about 20%, or from about 10% to about 19%, or from about 10% to about 18%, or from about 10% to about 17%, or from about 10% to about 16%, or from about 10% to about 15%, or from about 10% to about 14%, or from about 10% to about 13%, or from about 10% to about 12%, or from about 10% to about 11%, or from about 11% to about 25% (w/w), or from about 11% to about 24%, or from about 11% to about 23%, or from about 11% to about 22%, or from about 11% to about 21%, or from about 11% to about 20%, or from about 11% to about 19%, or from about 11% to about 18%, or from about 11% to about 17%, or from about 11% to about 16%, or from about 11% to about 15%, or from about 11% to about 14%, or from about 11% to about 13%, or from about 11% to about 12%, or from about 12% to about 25% (w/w), or from about 12% to about 24%, or from about 12% to about 23%, or from about 12% to about 22%, or from about 12% to about 21%, or from about 12% to about 20%, or from about 12% to about 19%, or from about 12% to about 18%, or from about 12% to about 17%, or from about 12% to about 16%, or from about 12% to about 15%, or from about 12% to about 14%, or from about 12% to about 13%, or from about 13% to about 25% (w/w), or from about 13% to about 24%, or from about 13% to about 23%, or from about 13% to about 22%, or from about 13% to about 21%, or from about 13% to about 20%, or from about 13% to about 19%, or from about 13% to about 18%, or from about 13% to about 17%, or from about 13% to about 16%, or from about 13% to about 15%, or from about 13% to about 14%, or from about 14% to about 25% (w/w), or from about 14% to about 24%, or from about 14% to about 23%, or from about 14% to about 22%, or from about 14% to about 21%, or from about 14% to about 20%, or from about 14% to about 19%, or from about 14% to about 18%, or from about 14% to about 17%, or from about 14% to about 16%, or from about 14% to about 15%, or from about 15% to about 25% (w/w), or from about 15% to about 24%, or from about 15% to about 23%, or from about 15% to about 22%, or from about 15% to about 21%, or from about 15% to about 20%, or from about 15% to about 19%, or from about 15% to about 18%, or from about 15% to about 17%, or from about 15% to about 16%, or from about 16% to about 25% (w/w), or from about 16% to about 24%, or from about 16% to about 23%, or from about 16% to about 22%, or from about 16% to about 21%, or from about 16% to about 20%, or from about 16% to about 19%, or from about 16% to about 18%, or from about 16% to about 17%, or from about 17% to about 25% (w/w), or from about 17% to about 24%, or from about 17% to about 23%, or from about 17% to about 22%, or from about 17% to about 21%, or from about 17% to about 20%, or from about 17% to about 19%, or from about 17% to about 18%, or from about 18% to about 25% (w/w), or from about 18% to about 24%, or from about 18% to about 23%, or from about 18% to about 22%, or from about 18% to about 21%, or from about 18% to about 20%, or from about 18% to about 19%, or from about 19% to about 25% (w/w), or from about 19% to about 24%, or from about 19% to about 23%, or from about 19% to about 22%, or from about 19% to about 21%, or from about 19% to about 20%, or from about 20% to about 25% (w/w), or from about 20% to about 24%, or from about 20% to about 23%, or from about 20% to about 22%, or from about 20% to about 21%, or from about 21% to about 25% (w/w), or from about 21% to about 24%, or from about 21% to about 23%, or from about 21% to about 22%, or from about 22% to about 25% (w/w), or from about 22% to about 24%, or from about 22% to about 23%, or from about 23% to about 25% (w/w), or from about 23% to about 24%, or from about 24% to about 25% (w/w), of the composition. Preferred embodiments may comprise from about 4.3%, 5%, 6.3%, 8.5%, 10%, 11.2%, 12%, 15%, and 15.2% w/w.

The composition of the present invention may comprise a number of ingredients, which include:

### Abrasives

According to an embodiment, the personal care composition of the present invention may contain an abrasive in addition to the cuttlefish bone powder used in the present invention. Preferably, the abrasive is chosen from colloidal calcium or colloidal silica. Suitable abrasive include hydrated silica and sodium bicarbonate (NaHCO₃). Other suitable abrasives include but are not limited to aluminum hydroxide (Al(OH)₃), calcium carbonate (CaCO₃), various calcium hydrogen phosphates (CaHPO₄.2H₂O, or anhydrous), various silicas (such as fumed silica, precipitated silica) and zeolites, and hydroxyapatite (Ca₅(PO₄)₃OH). Abrasive are insoluble particles that help remove tartar (plaque) from the teeth, and help remove dead cells from the skin. In toothpaste systems, the abrasive silica was shown to be the principal tooth cleaning and abrasive agent.

According to an embodiment abrasives may constitute from about 0.100% to about 0.325%, or from about 0.100% to about 0.300%, or from about 0.100% to about 0.275%, or from about 0.100% to about 0.250%, or from about 0.100% to about 0.225%, or from about 0.100% to about 0.200%, or from about 0.100% to about 0.175%, or from about 0.100% to about 0.150%, or from about 0.100% to about 0.125%, or 0.125% to about 0.325%, or from about 0.125% to about 0.300%, or from about 0.125% to about 0.275%, or from about 0.125% to about 0.250%, or from about 0.125% to about 0.225%, or from about 0.125% to about 0.200%, or from about 0.125% to about 0.175%, or from about 0.125% to about 0.150%, or 0.150% to about 0.325%, or from about 0.150% to about 0.300%, or from about 0.150% to about 0.275%, or from about 0.150% to about 0.250%, or from about 0.150% to about 0.225%, or from about 0.150% to about 0.200%, or from about 0.150% to about 0.175%, or 0.175% to about 0.325%, or from about 0.175% to about 0.300%, or from about 0.175% to about 0.275%, or from about 0.175% to about 0.250%, or from about 0.175% to about 0.225%, or from about 0.175% to about 0.200%, or 0.200% to about 0.325%, or from about 0.200% to about 0.300%, or from about 0.200% to about 0.275%, or from about 0.200% to about 0.250%, or from about 0.200% to about 0.225%, or 0.225% to about 0.325%, or from about 0.225% to about 0.300%, or from about 0.225% to about 0.275%, or from about 0.225% to about 0.250%, or 0.250% to about 0.325%, or from about 0.250% to about 0.300%, or from about 0.250% to about 0.275%, or 0.275% to about 0.325%, or from about 0.275% to about 0.300%, or 0.300% to about 0.325% (w/w) of the composition of the present invention. According to an embodiment, the colloidal calcium may be from about 0.100% to about 0,275% (w/w) of the composition. According to another embodiment, the colloidal silica may be from about 0.02% to about 0.08% (w/w) of the composition.

### Thickening Agents

According to an embodiment, the personal care composition of the present invention may contain a thickening agent.

Thickening agents, or thickeners, are substances which increase the viscosity of a solution or liquid/solid mixture without substantially modifying its other properties; although most frequently applied to foods where the target property is taste, the term also is applicable to paints, inks, explosives, etc. Thickeners may also be referred to as “natural gums”. Thickeners may also improve the suspension of other ingredients or emulsions which increases the stability of the product. Thickening agents are often regulated as food additives and as cosmetics and personal hygiene product ingredients. Some thickening agents are gelling agents (gallants), forming a gel, dissolving in the liquid phase as a colloid mixture that forms a weakly cohesive internal structure. Examples of suitable thickeners include but are not limited to natural gums obtained from seaweeds, such as agar (E406), alginic acid (E400) and Sodium alginate (E401), potassium alginate, ammonium alginate, calcium alginate, carrageenan (E407); natural gums obtained from non-marine botanical resources, acacia gum, gum arabic (E414), gum ghatti, gum tragacanth (E413), karaya gum (E416), guar gum (E412), locust bean gum (E410), beta-glucan, chicle gum, dammar gum, Glucomannan (E425), mastic gum, psyllium seed husks, spruce gum, tara gum (E417); natural gums produced by bacterial fermentation: gellan gum (E418), Xanthan gum (E415).

Also included are starches, pectins, carboxymethyl celluloses, hydroxypropyl celluloses, methyl cellulose and gelatin. Cellulose gum is the common name for carboxymethylcellulose, or CMC. Its emulsifying properties make it especially useful for products with ingredients that tend to separate, such as yogurt and jellies. Its ability to bind water makes it especially useful for diet foods, which tend to substitute water or other liquids for fat. Cellulose gum also improves texture, so it is a common ingredient in ice cream and frosting, products in which smoothness is a mark of quality. Beer manufacturers also use cellulose gum to stabilize beer foam. These same properties are useful for some pharmaceutical products that tend to separate over time, such as toothpaste. In the cosmetics industry, cellulose gum appears in bath products, makeup, shaving gels and hair products. According to an embodiment, the preferred thickening agents include but are not limited to xanthan gum, carboxymethylcellulose, and guar gum.

According to another embodiment, the thickening agent may be present in the formulation in about 2% to about 66% (w/w), or from about 5% to about 66% (w/w), or from about 10% to about 66% (w/w), or from about 15% to about 66% (w/w), or from about 20% to about 66% (w/w), or from about 25% to about 66% (w/w), or from about 30% to about 66% (w/w), or from about 35% to about 66% (w/w), or from about 40% to about 66% (w/w), or from about 45% to about 66% (w/w), or from about 50% to about 66% (w/w), or from about 55% to about 66% (w/w), or from about 60% to about 66% (w/w), or from about 2% to about 60% (w/w), or from about 5% to about 60% (w/w), or from about 10% to about 60% (w/w), or from about 15% to about 60% (w/w), or from about 20% to about 60% (w/w), or from about 25% to about 60% (w/w), or from about 30% to about 60% (w/w), or from about 35% to about 60% (w/w), or from about 40% to about 60% (w/w), or from about 45% to about 60% (w/w), or from about 50% to about 60% (w/w), or from about 55% to about 60% (w/w), or from about 2% to about 55% (w/w), or from about 5% to about 55% (w/w), or from about 10% to about 55% (w/w), or from about 15% to about 55% (w/w), or from about 20% to about 55% (w/w), or from about 25% to about 55% (w/w), or from about 30% to about 55% (w/w), or from about 35% to about 55% (w/w), or from about 40% to about 55% (w/w), or from about 45% to about 55% (w/w), or from about 50% to about 55% (w/w), or from about 2% to about 50% (w/w), or from about 5% to about 50% (w/w), or from about 10% to about 50% (w/w), or from about 15% to about 50% (w/w), or from about 20% to about 50% (w/w), or from about 25% to about 50% (w/w), or from about 30% to about 50% (w/w), or from about 35% to about 50% (w/w), or from about 40% to about 50% (w/w), or from about 45% to about 50% (w/w), or from about 2% to about 45% (w/w), or from about 5% to about 45% (w/w), or from about 10% to about 45% (w/w), or from about 15% to about 45% (w/w), or from about 20% to about 45% (w/w), or from about 25% to about 45% (w/w), or from about 30% to about 45% (w/w), or from about 35% to about 45% (w/w), or from about 40% to about 45% (w/w), or from about 2% to about 40% (w/w), or from about 5% to about 40% (w/w), or from about 10% to about 40% (w/w), or from about 15% to about 40% (w/w), or from about 20% to about 40% (w/w), or from about 25% to about 40% (w/w), or from about 30% to about 40% (w/w), or from about 35% to about 40% (w/w), or from about 2% to about 35% (w/w), or from about 5% to about 35% (w/w), or from about 10% to about 35% (w/w), or from about 15% to about 35% (w/w), or from about 20% to about 35% (w/w), or from about 25% to about 35% (w/w), or from about 30% to about 35% (w/w), or from about 2% to about 30% (w/w), or from about 5% to about 30% (w/w), or from about 10% to about 30% (w/w), or from about 15% to about 30% (w/w), or from about 20% to about 30% (w/w), or from about 25% to about 30% (w/w), or from about 2% to about 25% (w/w), or from about 5% to about 25% (w/w), or from about 10% to about 25% (w/w), or from about 15% to about 25% (w/w), or from about 20% to about 25% (w/w), or from about 2% to about 20% (w/w), or from about 5% to about 20% (w/w), or from about 10% to about 20% (w/w), or from about 15% to about 20% (w/w), or from about 2% to about 15% (w/w), or from about 5% to about 15% (w/w), or from about 10% to about 15% (w/w), or from about 2% to about 10% (w/w), or from about 5% to about 10% (w/w), or from about 2% to about 5% (w/w). According to an embodiment, the concentration is about 2.2% (w/w).

### Humectants

According to another embodiment, the composition of the present invention may further comprise a humectant. Humectants are substance used to keep things moist. When used as a food additive, the humectant has the effect of keeping the foodstuff moist. Humectants are also found in many cosmetic products where moisturization is desired, including treatments such as moisturizing hair conditioners and also commonly used in body lotions. Examples of humectants include but are not limited to propylene glycol, as well as hexylene glycol and butylene glycol, glyceryl triacetate, vinyl alcohol, neoagarobiose, sugar polyols such as glycerol, sorbitol, xylitol and maltitol, polymeric polyols like polydextrose, polyethylene glycol, polypropylene glycol, and poly(tetramethylene ether) glycol, quillaia, lactic acid, urea, glycerin, aloe vera gel, MP Diol, alpha hydroxy acids like lactic acid, and honey. According to another embodiment, the preferred humectant may glycerin.

According to another embodiment of the present invention, the humectant may be from about 2% to about 5% (w/w), or from about 2% to about 4% (w/w), or from about 2% to about 3% (w/w), or from about 3% to about 5% (w/w), or from about 3% to about 4% (w/w), or from about 4% to about 5% (w/w) of the composition.

### Emulsifier

According to an embodiment, the composition of the present invention may further comprise an emulsifier. An emulsifier is a substance that stabilizes an emulsion by increasing its kinetic stability. According to an embodiment, the emulsifier may be a lecithin, a vegetal pulp powder (such as citrus pulp powder, baobab pulp powder, mango pulp powder, tomato pulp powder, pumpkin pulp powder, guava pulp powder, papaya pulp powder and beet pulp powder), sodium citrate (e.g. trisodium citrate) and citric acid. The preferred emulsifier is sodium citrate.

According to another embodiment of the present invention, the emulsifier may be from about 4% to about 10% (w/w) or from about 4% to about 9%, or from about 4% to about 8%, or from about 4% to about 7%, or from about 4% to about 6%, or from about 4% to about 5%, or from about 5% to about 10%, or from about 5% to about 9%, or from about 5% to about 8%, or from about 5% to about 7%, or from about 5% to about 6%, or from about 6% to about 10%, or from about 6% to about 9%, or from about 6% to about 8%, or from about 6% to about 7%, or from about 7% to about 10%, or from about 7% to about 9%, or from about 7% to about 8%, or from about 8% to about 10%, or from about 8% to about 9%, or from about 9% to about 10% of the composition.

### Surfactants

According to an embodiment, the composition of the present invention may further comprise a surfactant. Surfactants are often, but always included in toothpaste and other oral care compositions. For example, toothpastes may contain sodium lauryl sulfate (SLS, also known as sodium dodecyl sulfate, SDS) or related surfactants (detergents). SLS is found in many other personal care products, as well, such as shampoo, and is mainly a foaming agent, which enables uniform distribution of toothpaste, improving its cleansing power. Other suitable surfactants include, but are not limited to ammonium lauryl sulfate, sodium N-lauryl sarcosinate (also known as sodium sarcosinate, and sodium lauryl sarcosinate) and sodium lauryl sulfoacetate.

Surfactants (detergents) also help clean the teeth, and provide foam that helps to carry away debris. Moreover, lauryl sulfates have significant anti-bacterial properties, and they can penetrate and dissolve plaque.

According to an embodiment, the surfactant may be from about 1% to about 3% (w/w), or from about 2% to about 3% (w/w), or from about 1% to about 2% (w/w) of surfactant.

### pH Regulator

According to an embodiment, the compositions of the present invention may contain a pH regulator. The product pH influences its stability and quality. When the pH is very acid, demineralization is favored, but if it is too basic, calcareous (tartar) deposits on the tooth can become important. Thus, the pH is preferably close to neutral pH, for example from about 6 to about 8, or from about 6.5, to about 7.5, or from about 6.75 to about 7.25, or about 7.0. The measured pH of the product is close to 6.8, or more specifically 6.78.

In embodiment, the pH regulator is an acid or a base which when added to the formulation stabilizes the pH at a desired level suitable for the oral care formulation of the present invention. Suitable pH regulator include but are not limited to citric acid and its derivatives, phosphoric acid and its derivatives, trisodium phosphate, sodium citrate, lactic acid, bicarbonic acid. The pH regulator may be present in concentrations of about 0.1% to about 0.28% (w/w), or from about 0.1% to about 0.25%, or from about 0.1% to about 0.2%, or from about 0.1% to about 0.15%, or from about 0.1% to about 0.12%, or about 0.12% to about 0.28%, or from about 0.12% to about 0.25%, or from about 0.12% to about 0.2%, or from about 0.12% to about 0.15%, or about 0.15% to about 0.28%, or from about 0.15% to about 0.25%, or from about 0.15% to about 0.2%, or about 0.2% to about 0.28%, or from about 0.2% to about 0.25%, or about 0.25% to about 0.28% (w/w) of the composition.

### Preservative

According to an embodiment, the compositions of the present invention may contain preservative agent. According to an embodiment, the preservative agent may sometime also act as an active antimicrobial agent for having an active role in the use of the composition.

Microorganisms can feed on humectants and thickening agents and ingredients to restrict their growth may be present in toothpaste. Generally, this is accomplished through minimal water and use of preservatives in the formulation. The most common preservatives in toothpaste are sorbitan sesquioleate derivatives, sodium benzoate, and benzoic acid. However, the compositions of the present invention may also be formulated with natural ingredients with preservative qualities or non-synthetic versions of common preservatives. Examples of natural products having preservative qualities include but is not limited to eucalyptus extract, essential oil having natural antimicrobial properties, such as eucalyptus oil, thyme oil, oregano oil, lemon oil, orange oil, and the likes, as well as natural antimicrobial agents such as thymol, carvacrol, eugenol, eucalyptol, menthol, etc., which are contained in these essential oils, or may be provided as isolated compounds. The composition may contain from about 0.2% to about 2%, and preferably about 0.5% w/w preservative.

### Solvents

According to another embodiment of the present invention, the compositions may comprise suitable solvents to formulate the compositions as mouthwashes, for example. Suitable solvents include but are not limited to water, ethanol, isopropanol, sorbitol and glycerin.

According to an embodiment, the composition may contain from about 60% to about 99% (w/w) of the solvent, or from about 70% to about 99% (w/w), or from about 80% to about 99% (w/w), or from about 90% to about 99% (w/w) of the solvent.

### Antimicrobial Agents

Antimicrobial agents that are useful in the present invention are the so-called “natural” antimicrobial actives. Such antimicrobial agents include natural essential oils and the individual antimicrobial compounds comprised in these oils. These actives derive their names from their natural occurrence in plants. Essential oils include oils derived from herbs, flowers, trees, and other plants. Such oils are typically present as tiny droplets between the plant's cells, and can be extracted by several methods known to those of skill in the art (e.g., steam distillation, enfleurage (i.e., extraction using fat(s)), maceration, solvent extraction, or mechanical pressing). Essential oils are typically named by the plant or vegetable in which the oil is found. For example, rose oil or peppermint oil is derived from rose or peppermint plants, respectively. Non-limiting examples of essential oils that can be used in the context of the present invention include oils of anise, lemon oil, orange oil, oregano, rosemary oil, wintergreen oil, thyme oil, lavender oil, clove oil, hops, tea tree oil, citronella oil, wheat oil, barley oil, lemongrass oil, cedar leaf oil, cedar wood oil, cinnamon oil, fleagrass oil, geranium oil, sandalwood oil, violet oil, cranberry oil, eucalyptus oil, vervain oil, peppermint oil, gum benzoin, basil oil, fennel oil, fir oil, balsam oil, menthol, ocmea origanum oil, Hydastis carradensis oil, Berberidaceae daceae oil, Ratanhiae and Curcuma longa oil, sesame oil, macadamia nut oil, evening primrose oil, Spanish sage oil, Spanish rosemary oil, coriander oil, thyme oil, pimento berries oil, rose oil, bergamot oil, rosewood oil, chamomile oil, sage oil, clary sage oil, cypress oil, sea fennel oil, frankincense oil, ginger oil, grapefruit oil, jasmine oil, juniper oil, lime oil, mandarin oil, marjoram oil, myrrh oil, neroli oil, patchouli oil, pepper oil, black pepper oil, petitgrain oil, pine oil, rose otto oil, spearmint oil, spikenard oil, vetiver oil, or ylang ylang. Other essential oils known to those of skill in the art are also contemplated as being useful within the context of the present invention (e.g., International Cosmetic Ingredient Dictionary, 10th edition, 2004, which is incorporated by reference). Also included in this class of essential oils are the key chemical components of the plant oils that have been found to provide the antimicrobial benefit (e.g., antimicrobial phenolic compounds).

The antimicrobial phenolic compounds of natural origin as used in the present invention can be synthetically made by known methods within the capacity of a skilled technician, or can be obtained from plant oil extracts. In an embodiment of the present invention, the phenolic compounds of natural origin are obtained from plant extracts. In a further embodiment of the present invention, the phenolic compounds of natural origin are commercially available. In yet further embodiments of the present invention, the phenolic compounds of natural origin comprise carvacrol, thymol, eugenol, eucalyptol, menthol, etc.

In an embodiment, the disinfectant formulations of the present invention comprise thymol, carvacrol or mixtures thereof. In a further embodiment, the disinfectant formulations of the present invention comprise one or more natural essential oils enriched in thymol, carvacrol or mixtures of thymol and carvacrol.

The compositions of the present inventions may contain from about 0.01% to about 10% (w/w), or from about 0.01% to about 9% (w/w), or from about 0.01% to about 8% (w/w), or from about 0.01% to about 7% (w/w), or from about 0.01% to about 6% (w/w), or from about 0.01% to about 5% (w/w), or from about 0.01% to about 4% (w/w), or from about 0.01% to about 3% (w/w), or from about 0.01% to about 2% (w/w), or from about 0.01% to about 1% (w/w), or from about 0.01% to about 0.75% (w/w), or from about 0.01% to about 0.5% (w/w), or from about 0.01% to about 0.25% (w/w), or from about 0.01% to about 0.10% (w/w), or from about 0.10% to about 10% (w/w), or from about 0.10% to about 9% (w/w), or from about 0.10% to about 8% (w/w), or from about 0.10% to about 7% (w/w), or from about 0.10% to about 6% (w/w), or from about 0.10% to about 5% (w/w), or from about 0.10% to about 4% (w/w), or from about 0.10% to about 3% (w/w), or from about 0.10% to about 2% (w/w), or from about 0.10% to about 1% (w/w), or from about 0.10% to about 0.75% (w/w), or from about 0.10% to about 0.5% (w/w), or from about 0.10% to about 0.25% (w/w), or from about 0.25% to about 10% (w/w), or from about 0.25% to about 9% (w/w), or from about 0.25% to about 8% (w/w), or from about 0.25% to about 7% (w/w), or from about 0.25% to about 6% (w/w), or from about 0.25% to about 5% (w/w), or from about 0.25% to about 4% (w/w), or from about 0.25% to about 3% (w/w), or from about 0.25% to about 2% (w/w), or from about 0.25% to about 1% (w/w), or from about 0.25% to about 0.75% (w/w), or from about 0.25% to about 0.5% (w/w), or from about 0.50% to about 10% (w/w), or from about 0.50% to about 9% (w/w), or from about 0.50% to about 8% (w/w), or from about 0.50% to about 7% (w/w), or from about 0.50% to about 6% (w/w), or from about 0.50% to about 5% (w/w), or from about 0.50% to about 4% (w/w), or from about 0.50% to about 3% (w/w), or from about 0.50% to about 2% (w/w), or from about 0.50% to about 1% (w/w), or from about 0.50% to about 0.75% (w/w), or from about 0.75% to about 10% (w/w), or from about 0.75% to about 9% (w/w), or from about 0.75% to about 8% (w/w), or from about 0.75% to about 7% (w/w), or from about 0.75% to about 6% (w/w), or from about 0.75% to about 5% (w/w), or from about 0.75% to about 4% (w/w), or from about 0.75% to about 3% (w/w), or from about 0.75% to about 2% (w/w), or from about 0.75% to about 1% (w/w), or from about 1% to about 10% (w/w), or from about 1% to about 9% (w/w), or from about 1% to about 8% (w/w), or from about 1% to about 7% (w/w), or from about 1% to about 6% (w/w), or from about 1% to about 5% (w/w), or from about 1% to about 4% (w/w), or from about 1% to about 3% (w/w), or from about 1% to about 2% (w/w), or from about 2% to about 10% (w/w), or from about 2% to about 9% (w/w), or from about 2% to about 8% (w/w), or from about 2% to about 7% (w/w), or from about 2% to about 6% (w/w), or from about 2% to about 5% (w/w), or from about 2% to about 4% (w/w), or from about 2% to about 3% (w/w), or from about 3% to about 10% (w/w), or from about 3% to about 9% (w/w), or from about 3% to about 8% (w/w), or from about 3% to about 7% (w/w), or from about 3% to about 6% (w/w), or from about 3% to about 5% (w/w), or from about 3% to about 4% (w/w), or from about 4% to about 10% (w/w), or from about 4% to about 9% (w/w), or from about 4% to about 8% (w/w), or from about 4% to about 7% (w/w), or from about 4% to about 6% (w/w), or from about 4% to about 5% (w/w), or from about 5% to about 10% (w/w), or from about 5% to about 9% (w/w), or from about 5% to about 8% (w/w), or from about 5% to about 7% (w/w), or from about 5% to about 6% (w/w), or from about 6% to about 10% (w/w), or from about 6% to about 9% (w/w), or from about 6% to about 8% (w/w), or from about 6% to about 7% (w/w), or from about 7% to about 10% (w/w), or from about 7% to about 9% (w/w), or from about 7% to about 8% (w/w), or from about 8% to about 10% (w/w), or from about 8% to about 9% (w/w), or from about 9% to about 10% (w/w) of antibacterial ingredients.

### Flavoring and Sweeteners

The composition of the present invention may contain a flavoring ingredient. The flavoring ingredient may be orange flavoring, apple flavoring, grapefruit flavoring, pineapple flavoring, strawberry flavoring, raspberry flavoring, cranberry flavoring, lime flavoring, lemon flavoring, grape flavoring, peach flavoring, any other fruit flavoring, vanilla flavoring, chocolate flavoring, caramel flavoring, mint flavoring, bubble gum flavoring, or any combination thereof.

Sweeteners such as aspartame, stevia, acesulfame, sucralose, maleic acid, citric acid, and the likes may also be included in the compositions of the present invention.

### Other Components

The composition of the present invention may contain other non-active excipients such as pigments and coloring agents, for example titanium oxide or other suitable pigments such as lactoflavins, chlorophylls such as copper derivatives of chlorophylls, and hydrogenated castor oil.

### Viscosity of the Composition

According to an embodiment, the viscosity of the oral care composition of the present invention may be from about 17500 to about 35000 cps, preferably 28800 cps, measured at 20° C. in a Brookfield apparatus at 20 rpm. The viscosity of the composition must be so that it does not prevent a good flow and good rinsing. The product is fully soluble in water.

### Storage Stability

The stability of the product was measured at 20° C. and 4° C. for 3 months. The product is placed in an oven and the physical and chemical characteristics measured that compare to the initial values. When he shows no phase separation, change in color, odor or deposit, it is considered stable in storage for two years.

### Stability to Heat

The heat stability is carried out by the product in an oven at 45° C. for 45 days. it is verified that the physical and chemical parameters are identical to the initial values and the product has no phase change, color or odor. It is considered that the product is stable on storage in the heat.

### Density Measurement

Good density ensures gives a good texture to the product and influences it's holding in suspension and stability. The measured value is equal to 1.14 while the desired value is from 1.10 to 1.35.

The present invention will be more readily understood by referring to the following examples which are given to illustrate the invention rather than to limit its scope.

### EXAMPLES

The Examples that follow are illustrative of specific embodiments of the invention, and various uses thereof. They are set forth for explanatory purposes only, and are not to be taken as limiting the invention.

The purpose of this study is to assess the efficacy of a toothpaste containing cuttlefish bone powder in calculus removal compared to a commercial anti-calculus toothpaste (Colgate Total®) that contains synthetic calcium carbonate (CaCO₃) as abrasive. The CB and synthetic CaCO₃ powders have been characterized using scanning electronic microscopy (SEM), Fourier transform infrared spectroscopy (FTIR), X-ray diffractometer (XRD) and zeta potential. On the other hand, the effect of toothpaste containing these powders on the enamel, dentin as well as calculus removal have been studied by mean of measuring the abrasion depth using stylus profilometer. Thus, there is a need for oral care formulations containing no harmful chemicals, providing efficient oral cleansing.

### Example 1

### Enamel/Dentin and Calculus Specimens Preparation

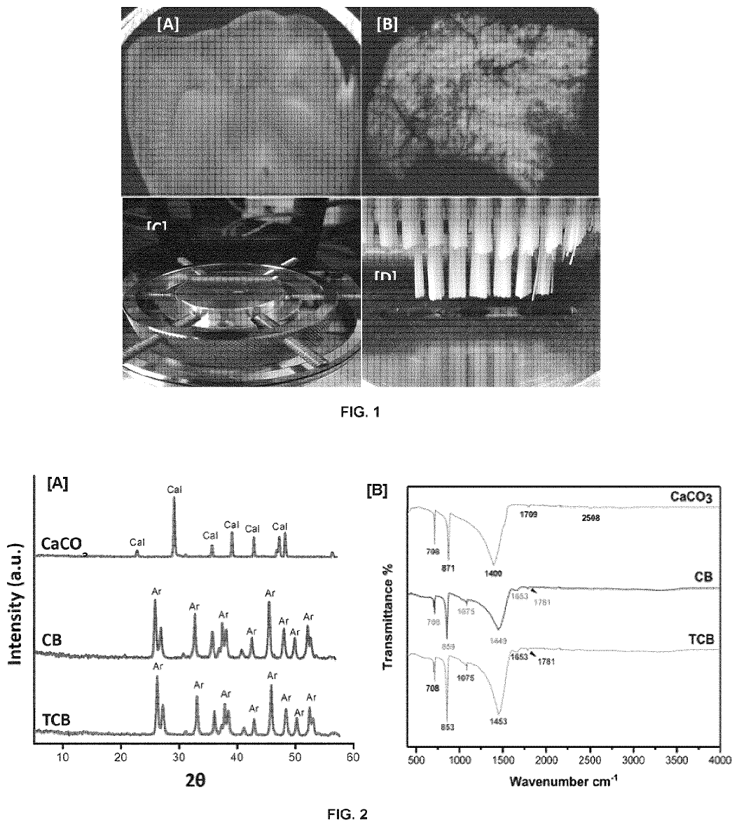

Enamel/dentin sections (4×4×3 mm) chipped from extracted teeth and natural calculus specimens collected from dental clinics were cleaned by 10 minutes ultrasonication in distilled water, and then embedded in acrylic resin (FIGS. 1A and 1B). Each resin block sample included a calculus and an enamel/dentine specimen embedded in acrylic resin. Resin blocks were then polished using an established polishing method until a smooth flat surface of calculus, dentin and enamel were created. Briefly, samples were polished using successive finer #600-1200 silicon carbide abrasive (Grit® C.-wt, AA abrasive Philadelphia, Pa.). For a smooth finish, colloidal silica suspension as polishing slurry (<0.06 μm; Master Met; Buehler, USA) on a two kinds of reusable polishing cloths (15-0.02 μm; TexMet C, 1-0.02 μm; ChemoMet) were used. The polished samples embedded in acrylic resin were then ultrasonically cleaned in distilled water for 20 min and dried in an oven at 40° C.

Preparation of an Abrasive Agent from Cuttlefish Bones

Cuttlebone samples imported from Senegal are cleaned to remove remaining flesh and dried at 50° C. to 3% water content. Dried cuttlebones are stored in a hopper equipped with a screw feeder and subsequently ground in an ultra-centrifugal type rotor mill and sieved with a sieve having a cut-off between 55 and 65 μm. On the one end, the coarse portion (65%) coming out of the sifter is returned to the feed hopper of the mill to be ground further. On the other end, the fine portion (representing the raw material for the formulation of the abrasive agent) is stored temporarily in a hopper.

Treated cuttlebone (TCB) powder, is produced in a double-walled impervious sealed reactor with a condenser and a vent. Firstly, the water preheated with steam is mixed with the bone powder using a solid-liquid premix pump. This step is to prevent the generation of bone dust in the reactor and clogging of the condenser as well as the loss of powder during changing of the reactor. Secondly, ammonium chloride is added to the reactor and viscosity of the clay is measured at 20° C. to be between 400 and 800 centipoises. The mass proportions were measured to be at 55% water, 35% bone powder and 10% ammonium chloride. Once this formulation is complete, the mixture is filtered in a basket centrifuge with porosity of 55 μm and washed with water until a neutral pH is obtained. Finally, the mix is dried in a tunnel dryer and the TCB is collected at the dryer outlet. A suitable preservative agent is added and homogenized with the powder in a double-cone mixer before being poured into a hopper and packaged in plastic bags.

Particle size of the TCB powder was analyzed using a Horiba LA-920 particle size analyzer. The sample of TCB was dispersed in isopropanol. Ultrasonicated for 1 min, and measured. Now referring to FIG. 18, there is shown the particle size distribution of TCB observed, having a % on Diameter as follows: D10=15.0886 μm, D50=46.6078 μm, D90=118.0572 μm. The mode of the distribution is 63.1988 μm, the mean (average) is 59.9973 μm and the median (middle value) is 48.6078 μm.

FIG. 18 shows that the TCB comprises a very small amount of (less than 1%) of very fine particles having diameters of less than 0.67 μm, and less than 4% of total particles having diameters equal to 8.8 μm or less. Particles of about 10 μm to about 20 μm represent about 12% of total particles, particles of above 20 μm up to about 30 μm represent about 13.8% of total particles, particles of above 30 μm up to about 40 μm represent about 10.7% of total particles, particles of above 40 μm up to about 45 μm represent about 5.8% of total particles, particles of above 45 μm up to about 60 μm represent about 12.9% of total particles, particles of above 60 μm up to about 68 μm represent about 6.9% of total particles, particles of above 68 μm up to about 77 μm represent about 6.8% of total particles, particles of above 77 μm up to about 89 μm represent about 6.4% of total particles, particles of above 89 μm up to about 101 μm represent about 5.6% of total particles, particles of above 101 μm up to about 116 μm represent about 4.6% of total particles, particles of above 116 μm up to about 133 μm represent about 3.5% of total particles, particles of above 133 μm up to about 152 μm represent about 2.5% of total particles, particles of above 152 μm up to about 175 μm represent about 1.7% of total particles, and particles of above 175 μm up to about 400 μm represent about 4.6% of total particles.

The CB sample was found to have a comparable size distribution, and the synthetic calcium carbonate (CaCO₃) had an average particle size of 26 μm.

### Example 2

### Toothpaste Compositions

A toothpaste composition is formulated for use in the current study.

The composition is as follow: Water (50%), treated cuttlefish bone powder (11%), sorbitol (20.74%), Glycerin (10%), Xylitol (10%), Silica (9%), Sodium Citrate (2.02%), Flavor (1.22%), Xanthan Gum (0.5%), Sodium Lauroyl Sarcosinate (0.9%), Titanium Dioxide (0.5%), Sodium Saccharin (0.25%), Cellulose Gum (0.5%), Menthol (0.06%), Mentha Viridis (Spearmint) Leaf Oil (0.01%), Citric Acid (0.05%), Phenoxyethanol (0.72%), Ethylhexylglycerin (0.08%).

### Example 3

### Brushing Test

To assess the abrasiveness of the toothpaste, a brushing test was established.

Briefly, the resin-embedded samples were mounted (FIGS. 1A and 1B) to a customized brushing machine (Mach-1, Biomomentum, QC) in a specifically designed mold that only exposed 0.5 mm×15 mm of the sample to the brush.

A customized toothbrush was fixed in the machine parallel to the sample surface (FIGS. 1C and 1D). Each toothpaste was diluted in distilled water at a ratio of 2:1 (w:w). The resulting slurries were then used to brush the blocks in the machine for 56 minutes at 90 strokes/min (amplitude of 10 mm) under a load of 500 g. This is equivalent to a regular tooth brushing of 2-minute per session, twice a day for 2 weeks.

### Example 4

### Abrasion Measurement Using Profilometer

To quantify the degree of surface wear, sample profile was measured on 3 lines for each sample across the area of sample that was exposed to the brush. The abrasion depth was measured using a profilometer (Dektak XT profilimometer) by referring to the sample surface that was not in touch with the brush as the baseline. The measurements shown are based on the deepest point in the sample profile when compared to the baseline. Samples of cuttlebone (CB) powder, TCB and synthetic CaCO₃ were studied by X-ray diffraction (XRD) analysis using a Bruker AXS GmbH, Karlsruhe, Germany diffractometer using a Cu K_{α} radiation source (λ K_{α}=1.5406 Å), and a monochromator operated at 40 kV and 40 mA, within the 10 to 60° range in 2θ in a step scan mode with steps of 0.02° and counting time of 4 s per step. This technique could to identify the crystalline nature of the samples.

To identify the surface functionalization, Infrared (IR) spectra of the samples were acquired using a Bruker™ Tensor 27 Fourier transform infrared (FTIR) spectrometer with accumulation of 64 scans in the range of 400-4000 cm⁻¹ at a resolution of 4 cm⁻¹. The morphology and microanalysis of the CB, TCB and synthetic CaCO₃ powder have been examined using an Inspect-50 field emission scanning electron microscope (SEM) (FEI, Japan) equipped with an energy dispersive X-ray spectrometer, at 5 kV operating voltage. To investigate the effect of toothpastes containing CB, TCB or CaCO₃ on the surface chemistry of embedded enamel/dentin, or calculus, a set of these samples have been immersed in solution containing 10 mg of CB, TCB or CaCO₃ in 50 ml doubly distilled (dd) H₂O for 20 minutes. The samples were washed with ddH₂O and dried. The change of surface chemistry of all samples was analyzed using Energy Diffraction X-Ray) (EDX). The surface charge of the CB, TCB or CaCO₃ was characterized by measurement of zeta potential. The zeta potential (z) of the CB, TCB or CaCO₃ powder at pH 7.5 was measured using a ZetaPALS (Brookhaven model BI-9000, Holtsville, N.Y.). To perform the measurements, 0.01 g of a powder sample was dispersed in 10 ml of deionized water. Then, 10 ml of the prepared sample was titrated either with acid (0.1M HCl) or with base (0.1M NaOH) for pH adjustment. For statistical analysis 3 samples of each hybrid powder was studied and the results derived were expressed as mean values±standard deviation.

Now referring to FIGS. 2A° and 2B, the results show that the main phase in both CB and TCB is Aragonite while the main phase in the CaCO₃ is Calcite. In comparison to the spectrum of the synthetic CaCO₃, the spectra of both CB and TCB show an additional amide I (1653 cm⁻¹) and a C═O group (1781 cm⁻¹). This band could reflect the organic nature of the cuttlefish bone powder. The chitin traces could endow the CB and TCB anti-inflammatory properties. FIG. 2B shows the FTIR spectra of the synthetic CaCO₃, cuttlefish bone (CB) powder and treated cuttlefish bone (TCB) powder. In the spectrum of synthetic CaCO₃ illustrated, the CaCO₃ has adsorption bands at 708, 871 and 1400 cm⁻¹ corresponding to the in-, out-plane bending and asymmetrical stretching vibration peaks of O—C—O, respectively. They all are characteristic peaks in calcite. The band near 1709 cm⁻¹ and 2508 cm⁻¹ can be attributed to the vibrations of the carbonate ions.

Both spectra of the CB and TCB show the characteristic bands of carbonate ions in the aragonitic structure at 708, 859 and 1449 cm⁻¹. While the band near 1781 cm⁻¹ in both spectra could be attributed to the vibrations of the carbonate ions. The observed absorption bands for aragonite phases (calcium carbonate) in cuttlebone structure are due to the planar CO₃² ion.

Theoretically, there are four vibrational modes in the free CO₃⁻² ion: the symmetric stretching vibration of the carbonate ion at about 1082 cm⁻¹ (v1); the out-of-plane bending absorption at about 857 cm⁻¹ (v2); the asymmetric stretching vibrations at about 1400-1500 cm⁻¹ (v3); and the split in-plane bending vibrations at about 700 cm⁻¹ (v4). In comparison to the spectrum of the synthetic CaCO₃, the spectra of CB and TCB show an additional band at 1653 cm⁻¹. This band is assigned to amide I band (two types of hydrogen bonds in a C═O group with the NH group of the adjacent chain and the OH group of the inter-chain). This band could reflect the organic nature of the cuttlefish bone powder.

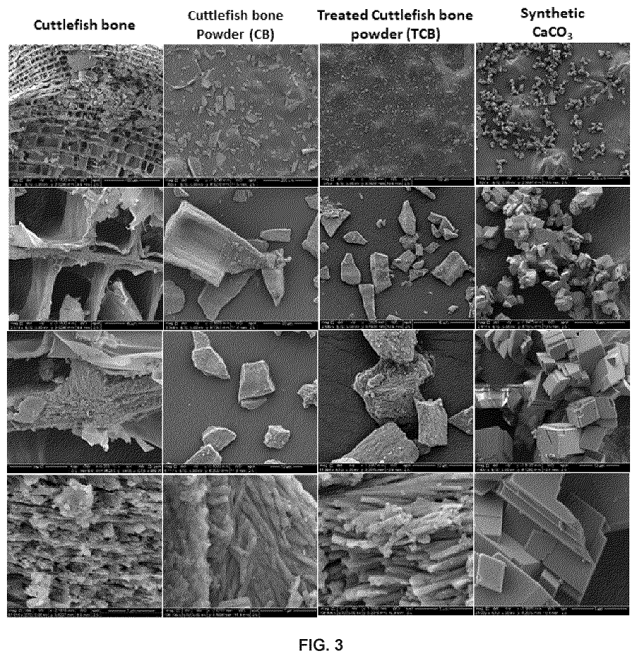

Now referring to FIG. 3. The Scanning electronic microscopy (SEM) micrographs of the synthetic CaCO₃, cuttlefish bone (CB) powder and treated cuttlefish bone (TCB) powder are shown in FIG. 3. The morphology of the cuttlefish bone reveals an approximately periodic microstructure. The CB perfectly preserved the morphology of the initial aragonitic cuttlefish bone. The lamellar matrix of CB consists of many horizontal thin sheets (lamellae) supported by transversal pillars that form chambers sealed from each other. The morphology of the grinded, CB powder and TCB powder show clusters with various shapes and sizes with an average size of about 10-30 μm. The morphology of the synthetic CaCO₃ powder shows the characteristics of calcite crystals. The synthetic CaCO₃ crystal particles are rectangular. The surfaces are smooth, and the edges and angles are sharp and clear. Many rhombohedron aggregate and wedge each other together.

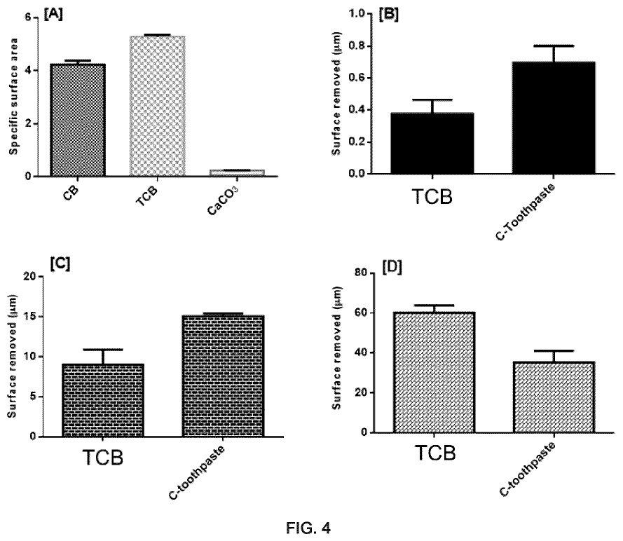

As shown in FIG. 3, the needle shaped particles CB and TCB have higher specific surface area values than the cubic forms of CaCO₃. The higher surface area makes the CB and TCB a promising anti calculus agent by covering higher area of the teeth surface. This agrees with the specific surface area measurements using Brunauer, Emmett and Teller (BET) theory which is shown in FIG. 4A. CB powder showed specific surface area of (4.230±0.1532), TCB was (5.290±0.0660) and CaCO₃ was (0.238±0.0077).

The results of abrasion depth based on brushing strokes were plotted and trend plot was obtained. FIGS. 4A and 4B show the abrasion depth of the proposed toothpaste containing CB (A) vs commercial toothpaste (B). Compared to the commercial toothpaste, the oral care composition according to the present invention showed lower abrasion depth for both dentin and enamel specimens (FIGS. 4B and C). This indicates that the oral care composition according to the present invention with TCB powder has less abrasivity over enamel and dentin than that of the commercial toothpaste. In terms of calculus removal, the abrasivity data shows that the oral care composition according to the present invention is more efficient in achieving mechanical removal of calculus when compared to commercial toothpaste (FIG. 4D). The effectiveness of the oral care composition according to the present invention, containing TCB may be due to the combination of TCB with higher surface area than synthetic CaCO₃ (FIG. 3) and the measured specific surface area as shown in FIG. 4A. These two parameters make such material more efficient in removing the calculus from rough surfaces.

Another parameter which could affect the performance of the oral care composition of the present invention is the hardness. The reported values of enamel's hardness ranges from 2.65 GPa to 3.53 GPa, from 0.49 GPa to 0.58 GPa for dentin, 20-90 Knoop hardness (KHN) for calculus, 5-5.5 GPa for synthetic CaCO₃ and cuttlefish bone has the highest mechanical hardness at 6.87-4.75 GPa. The unexpected enhancement of calculus removal in the case of the oral care composition according to the present invention containing TCB may be due to the higher hardness of the TCB powder particles in the oral care composition according to the present invention which consequently abrade the calculus more efficiently.

The results presented above suggest that the combination of hardness of TCB as well is its characteristic higher surface area (as shown in FIG. 4), unexpectedly lead the oral care composition according to the present invention to display superior abrasiveness on calculus than commercial toothpaste containing CaCO₃.

### Example 5

### Abrasion Quantification Using Profilometer

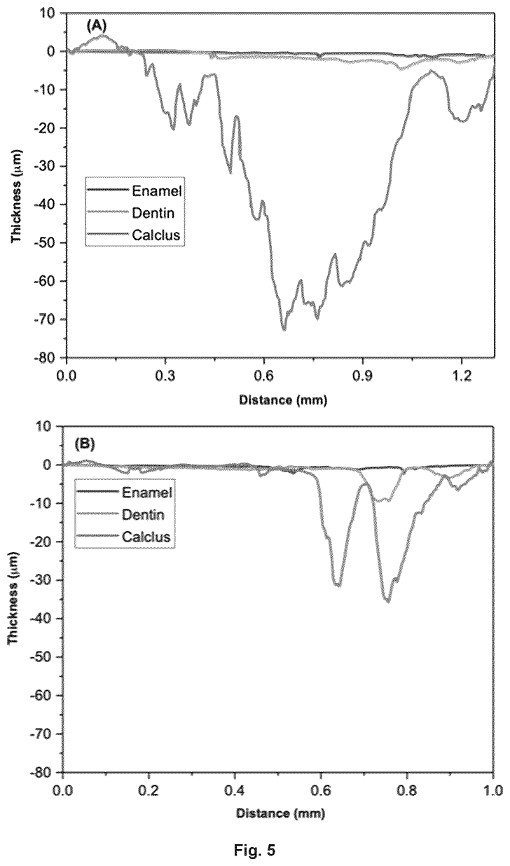

Now referring to FIG. 5. The surface profile method was useful in obtaining quantitative and qualitative results such as surface roughness on the brushed tooth surface (FIGS. 5A and 5B). This method quantifies the loss of calculus or dental tissue during the brushing process in relation to a non-treated reference area. Using profilometry and measuring the thickness of the removed layer in relation to a non-treated reference area showed that the calculus was more susceptible to abrasion using both an oral care composition according to the present invention (FIG. 5A) than commercial toothpastes (FIG. 5B). But the oral care composition according to the present invention is highly efficient in removing calculus while only slightly affecting the enamel and dentin. Therefore, the oral care composition according to the present invention containing the TCB can remove calculus mechanically but is also more gentle on the tooth enamel and dentin. It is therefore safer for long term use.

### Example 6

### Affinity of Teeth Components to Interact with the CB and CACO₃

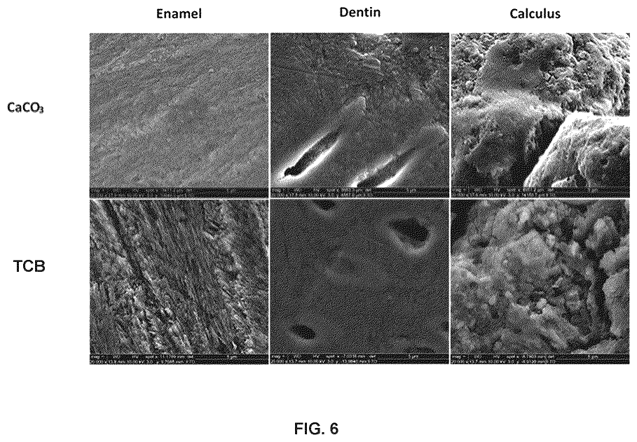

FIG. 6 shows the characteristic structure of the human enamel, dentin and calculus investigated on its cross section after immersion in solution containing CaCO₃ (Top) and TCB (bottom). In the human enamel studied, the basic microstructure blocks observed are enamel rods formed of structural hydroxyapatite needle-like crystallites composed mainly of calcium, phosphate and oxygen as indicated by EDX analysis (Table 1).

TABLE 1
EDX results of the surfaces of Enamel, Dentin and Calculus samples after
immersion in solution contain 10 mg of TCB or CaCO₃ for 30 minutes.

| | Enamel | | Dentin | | Calculus | |
|---|---|---|---|---|---|---|
| | EDX results | | | | | |
| | Before | After | Before | After | Before | After |
| Element | immersion | immersion | immersion | immersion | immersion | immersion |

TCB Natural

| | | | | | | |
|---|---|---|---|---|---|---|
| Cₖ | — | 5.56 ± 1.39 | 16.22 ± 0.47 | 15.40 ± 0.81 | 6.021 ± 043 | 11.60 ± 0.73 |
| Oₖ | 64.69 ± 1.22 | 53.4 ± 3.01 | 51.53 ± 1.55 | 52.18 ± 0.57 | 49.48 ± 2.11 | 50.47 ± 1.06 |
| Pₖ | 12.6 ± 0.70 | 12.11 ± 1.41 | 12.53 ± 0.91 | 12.51 ± 0.02 | 13.94 ± 1.01 | 9.29 ± 0.52 |
| Caₖ | 20.44 ± 0.66 | 21.23 ± 2.73 | 18.46 ± 1.77 | 17.25 ± 0.26 | 7.02 ± 0.2 | 11.83 ± 0.95 |

CaCO₃ Synthetic

| | | | | | | |
|---|---|---|---|---|---|---|
| Cₖ | — | 6.68 ± 84 | 16.68 ± 1.10 | 16.85 ± 1.57 | 14.19 ± 1.03 | 14.49 ± 0.24 |
| Oₖ | 54.66 ± 1.77 | 54.25 ± 2.06 | 47.39 ± 2.32 | 46.14 ± 3.57 | 56.27 ± 2.69 | 57.69 ± 0.70 |
| Pₖ | 13.37 ± 1.0 | 14.98 ± 1.04 | 14.34 ± 0.54 | 14.21 ± 1.87 | 11.52 ± 0.83 | 10.70 ± 0.22 |
| Caₖ | 21.11 ± 1.12 | 22.33 ± 1.55 | 20.69 ± 2.18 | 20.23 ± 2.62 | 13.16 ± 1.00 | 13.25 ± 0.37 |

The main structural features of dentin are the dentin tubules, which extend through the entire dentin thickness, but vary both in number and diameter along the thickness of the dentin. The dental calculus is a hard deposit that is formed by calcification of dental plaque primarily composed of calcium phosphate mineral salts as indicated by EDX analysis (Table 1), which is deposited on natural teeth and restorations and covered by a layer of unmineralized plaque.

After immersion in solutions containing TCB powder or synthetic CaCO₃, the dental calculus showed more affinity to interact with the TCB than with the synthetic CaCO₃. This is indicated by the increase of the carbon and reduction of phosphorus concentration after immersion of dental calculus in solution contain TCB powders. After immersion of dental calculus in solution contains synthetic CaCO₃, no significant change has been observed in the concentration of both carbon and phosphorus. This indicates that the TCB is more efficiently adsorbed on the surface of calculus than the synthetic CaCO₃.

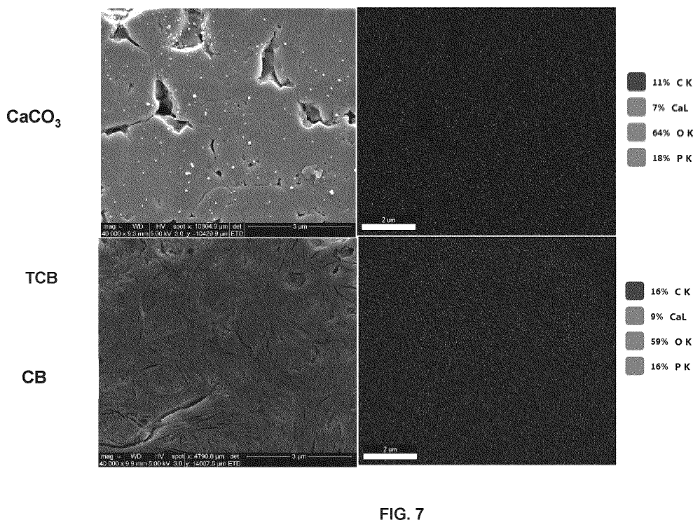

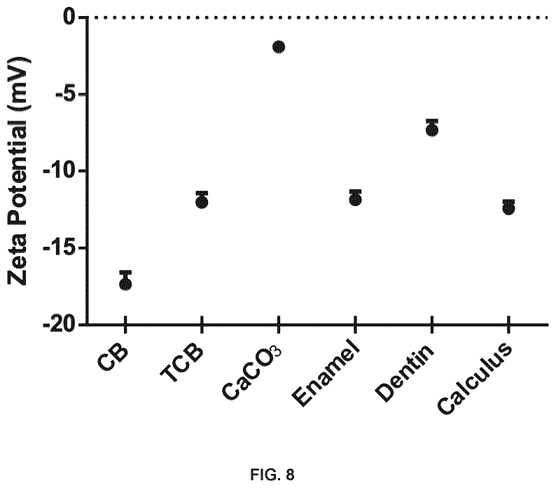

Now referring to FIG. 7. The zeta potential of human enamel is of physiological importance for interactions between teeth and the surrounding aqueous medium of saliva. The zeta potentials of enamel, dentin and calculus have been examined and compared to the zeta potential values of the CB and TCB and synthetic CaCO₃. The enamel particles were found to have strong negative surface potentials of −11.87±0.529 mV. Dentin particles exhibited a less negative zeta potential −7.33±0.6 mV. The calculus exhibited −12.45±0.452 mV more negative potential value than both dentin and enamel.

Both calcium and phosphate ions play a major role in determining the zeta potential, calcium ions making the zeta potential more positive and phosphate ions making it more negative. The results from the present study indicate the surface potentials of the calculus and enamel particles of −11 to −12 mV, and those for the dentin of −7 which are in agreement with the above statement. For the same reason the CB and TCB, with more phosphate, exhibited zeta potential of −17.35±0.78 and −12.04±0.598 both of which are more negative than that of the synthetic CaCO₃ which showed potential of −1.92±0.0688.

The in vitro results presented herewith show that the oral care composition of the present invention is a descaling dentifrice that effectively removes calculus attached on tooth surfaces after one month of brushing without causing damage to tooth surface. Moreover, the oral care composition of the present invention removes 5 times more calculus than commercial Colgate Total® toothpaste.

### Example 7

### Clinical Trial

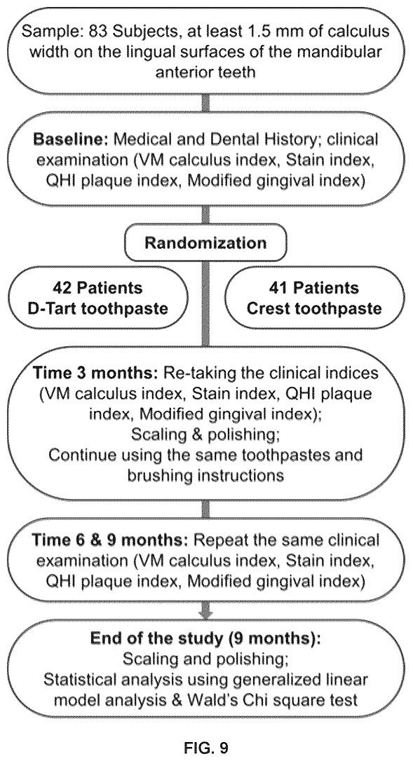

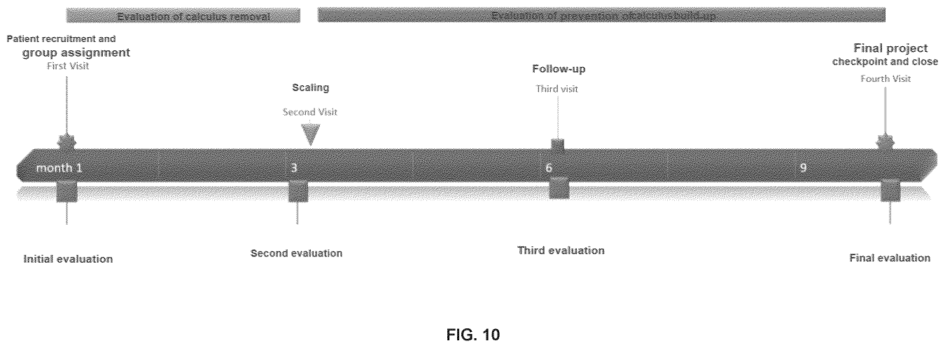

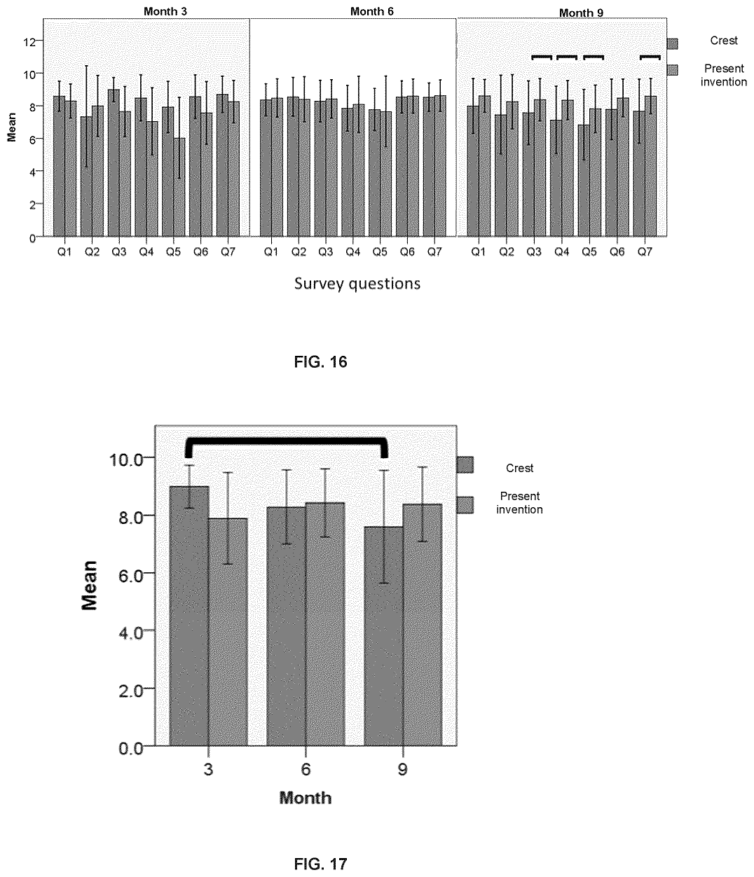

Now referring to FIGS. 9-17. A clinical trial comprising 83 subjects having at least 1.5 mm of calculus width on the lingual surfaces of the mandibular anterior teeth were subjected to brushing with an oral care composition according to the present invention (as described in Example 2). FIG. 9 illustrates the various steps of the trial while FIG. 10 presents a timeline of the trial. Table 2 provides information about the study groups.

TABLE 2
Study groups demographics

| | Invention | Commercial | | |
|---|---|---|---|---|
| Measurements | Group | product Group | Overall | p-value |
| Male | 14 (33%) | 12 (29%) | 25 | 0.16 |
| Female | 28 (67%) | 29 (71%) | 57 | |
| Age (years) | 44.5 ± 15.8 | 41.7 ± 14.6 | | NS |
| Ethnic Group | 40 (96%) | 40 (98%) | 80 | 0.57 |
| Caucasian | | | | |
| Ethnic Group | 2 (4%) | 1 (2%) | 3 | |
| Other | | | | |

This study was conducted as a double blind randomized controlled parallel-group trial. Intervention group received an oral care composition according to the present invention to remove calculus and stains on their teeth, while the control group received same treatment but using Crest® toothpaste. Crest® toothpaste was chosen because it has similar abrasiveness, pH and texture properties to an oral care composition according to the present invention, features that need to be controlled as to prevent unwanted interactions. For instance, abrasiveness can become a risk factor for oral problems if the abrasiveness of the toothpaste is too high, or the pH which it has been shown that the ingredients in toothpaste can act differently if the environment is basic or acidic.

Participants should meet the following criteria to be included in the study: 1) Aged 18 years and over, to ensure the compliance with the study instructions; 2) Systemically healthy to exclude the possibility of calculus formation due to disease; 3) Has at least 20 sound natural teeth including all lower anterior teeth, the main location of calculus build up; 4) Having the history of previous calculus formation (at least 1.5 mm of calculus width) on the lingual surfaces of the mandibular anterior teeth after 3-6 months of receiving a professional prophylaxis treatment, for better assessment of the toothpaste effectiveness; 6) Agree to follow the study instruction: adherence to study arm, for the study timeline.

### Exclusion Criteria

Subjects were excluded from participating in the study if they meet these exclusion criteria: 1) Pregnancy: pregnant women could not comply with long evaluation session or due to delivery and postpartum responsibilities. In addition, the gum inflammation that usually occurs during pregnancy could introduce bias to the study; 2) any physical handicap, psychological or health conditions that might jeopardize the ability of the patient to brush his/her teeth, these complications cause lack of motivation and bias in the study; 3) Antibiotics or anti-inflammatory drugs taken within 1 month prior to the study, to avoid bias when assessing the gum health; 4) Currently using Chlorhexidine oral products, since these products can affect biofilm and calculus formation, therefore they can introduce bias in our assessment of calculus removal by our toothpaste; 5) Sensitivity to tartar-control toothpastes; 6) Presence of oral prostheses, dental implants or fixed orthodontic appliances on teeth that will be included in the assessment, since these devices can introduce bias to the study because they tend to increase the rate of plaque accumulation; 7) Patients currently receiving dental treatment that would result in the removal of plaque or calculus and compromise our ability to measure calculus removed by the toothpastes; 8) unable to return for evaluations/study recalls, to avoid the significant loss in the number of participants; 9) Having the diagnosis of Periodontal Screening and Recording scale (PSR) of 4, these type of patients have advanced periodontitis with high risk of tooth loss (periodontal pockets of 6 mm and more) and could bias the treatment outcomes.

### Participants' Recruitment/Randomization

Participant who are interested in the study and fulfilled the selection criteria were asked to sign a consent form. Randomization was carried out off-site by a statistician at the Faculty of Dentistry at McGill University. After screening and clinical examination, all participants enrolled in the study were randomly assigned to receive either the intervention (oral care composition of the present invention) or control (Crest® toothpaste) groups following a computer-generated randomization list. The study was double blinded; oral care composition of the present invention and control toothpastes were provided in an identical packaging except for a coded identification number that was withheld from the examiners until data analysis is complete.

### Clinical Procedures and Evaluation

The primary outcome was the amount of calculus removed by the toothpastes. The secondary outcomes were the amount of extrinsic tooth stains removed from the upper and lower incisors, and reduction in gingival inflammation. All clinical parameters were evaluated by one calibrated examiner at baseline (first visit), 3, 6- and 9-months. At baseline session, the following clinical parameters were recorded:
1. The calculus accumulation on the lingual aspects of 6 mandibular anterior teeth using the Volpe-Manhold Calculus Index score.
2. The extrinsic stains on the labial and palatal/lingual surfaces of the upper and lower central and lateral incisors using the Shaw & Murray Stain Index].
3. The plaque accumulated on the labial and lingual surfaces of teeth using Quigley-Hain plaque index (QHI)
4. The gingival health; the buccal and lingual marginal gingiva and interdental papillae of all anterior teeth using the Modified Gingival Index (MGI)

After the initial evaluation, each participant was provided with the toothpaste and standard advice on tooth brushing (Modified Stillman brushing technique, brushing for 2 minutes, twice daily). Participants were instructed to not use mouthwashes or other toothpastes for the duration of the study. At 3 months, the participants were re-examined and the same data was recorded in order to evaluate the effectiveness of the toothpastes in removing the calculus and stains. In the same session, the participants were given a standard dental cleaning; thorough scale using ultrasonic and hand instruments followed by polishing with prophylaxis paste (Sunstar Butler, fine Bubble gum, Guelph, ON) and topical application of neutral fluoride. Then they were asked to continue using the same toothpaste and brushing technique/instructions described above. All the examinations were repeated for the participants at 6 and 9 months in order to evaluate the effectiveness of the toothpastes in preventing calculus formation. At the end of the trial (9-month visit), the participants were offered a scale and polish.

### Reliability of Measurements

One, well experienced dental hygienist was trained for the scoring system of all indices. The measurements were repeated for the first fifteen participants by the examiner to determine intra-examiner reliability. The examiners repeated the measurements at least one hour after the first one while being blinded to the data of the first measurements. The intra-examiner data were analyzed by calculation of Cohen's kappa test, which showed excellent agreement and significantly reliable results for all clinical measurements (κ=0.92-0.96).

### Patient Satisfaction Survey

A survey was performed at each one of the follow up appointments, (month 3, 6 and 9) the survey consisted of seven questions that were answered using a visual analog scale that ranges from highly satisfied to not satisfied at all. In addition, the survey had 2 additional open ended questions to register either “negative” or “positive” comments.

TABLE 3
Questions of the VAS questions in the patient satisfaction survey

| Question | |
|---|---|
| number | Question |
| Q1 | How satisfied you are with the cleaning effect of the toothpaste? |
| Q2 | How satisfied you are with the toothpaste taste? |
| Q3 | How satisfied you are with the toothpaste texture? |
| Q4 | How satisfied you are with the toothpaste consistence? |
| Q5 | How satisfied you are with the toothpaste stickiness? |
| Q6 | How satisfied you are with the toothpaste quality? |
| Q7 | How is your overall satisfaction with the current |
| | toothpaste compared to other brands? |

### Statistical Analysis

Sample size calculation: According to previous clinical trials studies comparing toothpastes for calculus removal, anti-calculus agents can reduce the amount of calculus by 30 to 50%. A sample size of 40 per group was required to detect a clinically relevant difference (4%) between test and control toothpastes that achieved a study power of 80% at a significant level of 0.05. To allow a 10% dropout, the final sample of 92 participants was recruited (Power and Sample Size Calculations software, Version 3.0, Vanderbilt University, Germany, using a two-sided independent samples t—test).

The normality of data distribution was initially tested. Calculus, stain, QHI and MGI scores were compared at 3, 6 and 9 months, using a generalized linear mixed models (GLMM) analysis with Wald's Chi-square test. Follow-up data was used as the dependent variable, the toothpaste as the fixed factor and the baseline scores as the covariate. P-values were considered statistically significant if less than 0.05. Comparison between groups regarding VAS scores for the satisfaction survey were done with student t-test and data was presented as mean and standard deviations, on the other hand analyses for complaints and compliments was done using Pearson Chi-square and data was presented as odd ratios.

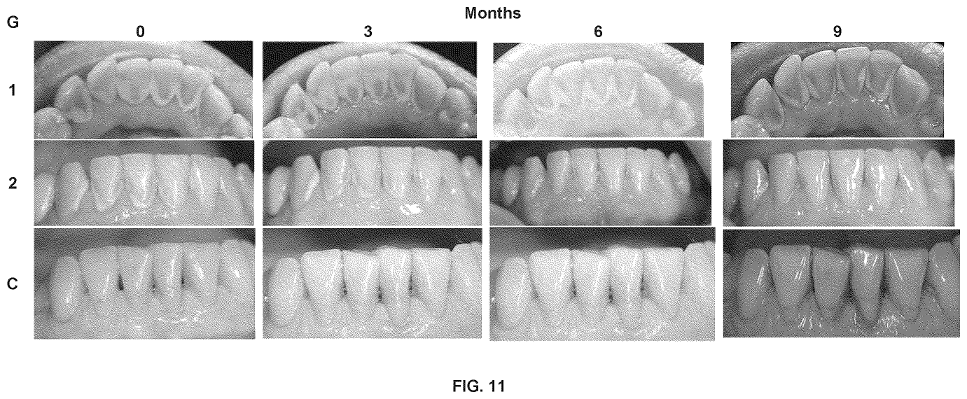

FIG. 11 shows teeth from clinical trial study participants treated with the oral care composition of the present invention. G is short for Group and 1 & 2 refer to the patients having received the oral care composition of the invention while C received the toothpaste (C).

### Clinical Parameters

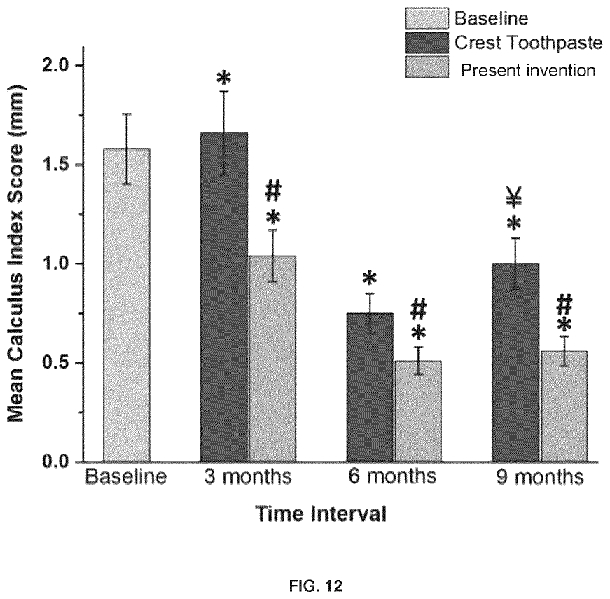

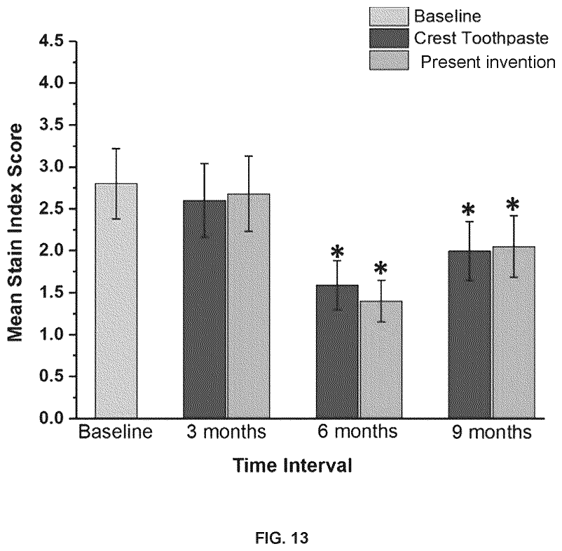

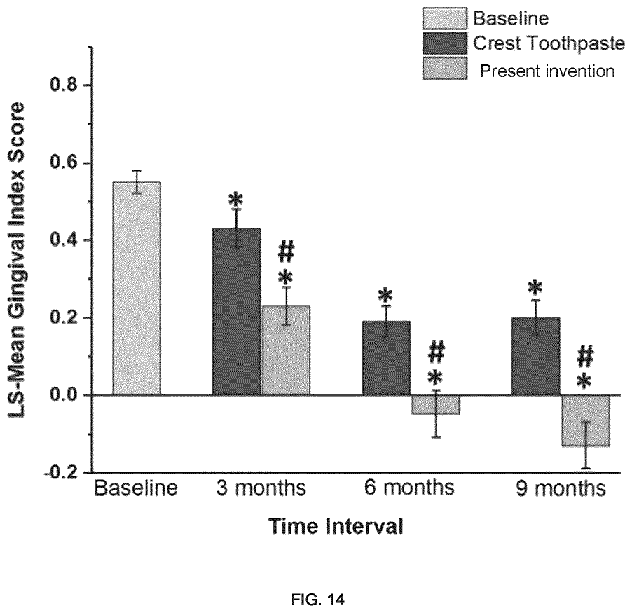

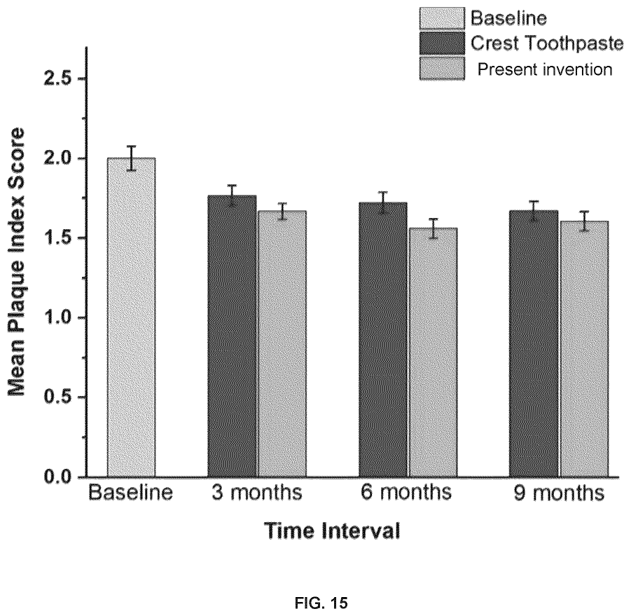

FIG. 12 illustrates the Volpe-Manhold Calculus Index before and after using the toothpastes. FIG. 13 illustrates the Shaw & Murray Stain Index before and after using the toothpastes. FIG. 14 illustrates the Modified Gingival Index before and after using the toothpastes. Scores are expressed as MI mean±S.E.M. *=significantly different from baseline score (before using the toothpastes); #=significantly different from Crest® toothpaste group. V=significantly different from time 6 months score (p<0.05. Crest® toothpaste=commercial product. FIG. 15 illustrates Quigley-Hain Plaque Index before and after using the toothpastes. In each case, the scores are expressed as MI mean±S.E.M. *=significantly different from baseline score (before using the toothpastes); #=significantly different from Crest® toothpaste group. V=significantly different from time 6 months score (p<0.05). Crest® toothpaste=commercial product.

### Calculus Level

At time 3 months, Crest® group showed 16.69% more calculus accumulation while the present invention showed 32.3% less calculus (Bonferroni-adjusted p=0.0007) in comparison to baseline level. Comparing the two groups at time 3 months showed that, the mean calculus score for Crest® group is 58.9% greater than that that for the present invention group (p=0.0001).

At time 6 and 9 months, both study groups showed significantly less calculus accumulation than that at baseline. Comparing the two groups at the same time intervals showed that the mean calculus level in the present invention group is (48.7%) and (78.7%) less than that in the Crest® group respectively (p=0.0001, 0.0008), indicating the efficacy of Crest® toothpaste in preventing calculus formation. Moreover, the level of calculus is significantly higher at time 9 months in comparison to that at 6 months interval for the Crest® group, however this increase in not significant for the present invention group.

### Extrinsic Stain

The mean stain score did not significantly change over the 3 months period in comparison to baseline (time 0) for both study groups, indicating that both toothpastes were not able to efficiently remove the stain in 3 months (p=0.49). However, at time 6 and 9 months, the mean stain score is significantly less than that at baseline for both study groups (p<0.0001). Moreover, there is no significant difference between study groups at both 6- and 9-months regarding mean stain score, indicating that the present invention toothpaste has the same efficacy of Crest® toothpaste in terms of stain removal (p=0.52 and 0.88 respectively).

### Gingival Health

At 3 months, the mean score of MGI for the present invention group was 37% lower than that recorded at baseline (p<0.0001) and 21% lower than that for Crest® group (p=0.0380), indicating the significant improvement of the present invention participants' gingival health after 3 months of use. Moreover, the mean score of MGI for the present invention group was 27% and 38% lower than that for Crest® group at time 6 months (p=0.0199) and time 9 (p=0.0003) retrospectively, indicating that the present invention toothpaste was significantly more effective than Crest® toothpaste in enhancing the gingival health.

### Plaque Accumulation

At 3 months, the mean score of plaque index was significantly higher than that recorded at baseline for both study groups (p<0.0001) with no significant difference in the mean plaque score between the two study groups (p=0.56), indicating that both toothpastes were similarly effective in removing plaque after 3 months of use.

Similarly, the mean score of plaque index at 6 months was significantly lower than that at baseline months with no further increase in the plaque level afterwards for both study groups, indicating that both toothpastes were significantly effective in preventing plaque accumulation (p<0.0001). Comparing the mean score of plaque index between the study groups showed no significant difference at 6 months (p=0.3) and 9 months (p=1).

### Safety and Patients' Satisfactions

The participants in both study groups experienced no adverse events during the study and both toothpastes were well tolerated. A total of 14 patients reported a negative comment in the control group compared to 9 in the present invention group; 6 patients reported tooth sensitivity in the control group compared to 2 patients in the present invention group, and 3 patients reported an incident of an oral ulcer in the control group compared to none in the present invention group. However, the differences between the control and experimental group in terms of negative events and complaints was not significant. On the other hand, patients in the present invention group reported a significantly higher number of compliments.

The patient satisfaction survey revealed no significant difference between groups after 3 and 6 months of using the toothpastes, however, after 9 months patients using the present invention toothpaste registered significantly higher satisfaction scores in 4 categories: texture, consistence and stickiness and overall satisfaction.

TABLE 4
Analysis of Patient’s’ comments at end of study

| | Control | Present | | |
|---|---|---|---|---|
| Comments | (ref) | invention | OR | P |
| Sensibility | 6/35 | 2/40 | 0.29 (95% CI:0.06-1.54) | 0.1465 |
| Ulcers | 3/38 | 0/42 | 0.129 (95% CI:0.01-2.59) | 0.1810 |
| Negative | 14/27 | 9/33 | 0.53 (95% CI:0.198 | 0.199 |
| comments | | | to 1.40) | |
| Compliments | 21/20 | 36/6 | 7.56 (95% CI:2.47 | 0.0007 |
| | | | to 23.13) | |

### DISCUSSION

The results presented herein indicated that at 3 months, the interventional group showed 32% less total calculus compared to the baseline mean score (p=0.0007) and 59% less total calculus compared to the control group (p=0.0001). Over the 9 months, the mean calculus score for Crest® users was 79% higher than that for the present invention users (p=0.0008). There was a significant improvement in the gingival health of the present invention users than the Crest® group at all intervals. However, both toothpastes were comparable in terms of stain removal, which was significantly lower than that at the baseline (p<0.0001).

### Calculus Removal

During the first 3 months of our study no scaling was performed, therefore, the changes in calculus buildup during this period are indicative of the calculus removal capabilities of the oral hygiene measurements of the patients. In the control group, there was no change in the amount of calculus, which indicates that the Crest® toothpaste was not able to remove calculus. This is consistent with the literature on toothpaste for calculus control. On the other hand, the patients that used the present invention toothpaste presented an unexpected significant reduction in calculus buildup. This result indicates that the present invention toothpaste is able to remove calculus in significant amounts. This is probably related to the unique hardness and specific surface area of the cuttlefish bone powder abrasives.

One concern of increased abrasiveness for calculus removal could be enamel and dentin wear. This is usually translated into increase sensitivity, however, the patients treated with the present invention toothpaste did not report this type of problem, with only 1 reported sensitivity issue in the control group and 2 in the present invention group.

### Stain Removal

Both toothpaste had a similar performance in terms of stain removal. This was expected as stains have a surface chemistry and topology that is different than that of calculus, and they are probably removed by the action of the emulsifiers in the toothpaste rather than by the abrasive action of the toothpaste thus the fact that the Crest® toothpaste is very well designed for this purpose and the prevention of calculus formation.

Interestingly, after scaling, the patients treated with the present invention toothpaste presented low levels of calculus over a prolonged period of time compared to the patients treated with the Crest® toothpaste who showed a progressive increase in calculus buildup over time.

### Strengths and Limitations

A main strength of this study is the randomization of the patients, which is confirmed by the similar baseline characteristics in both study groups. Another very important strength is the blinding at multiple levels; the patient was blinded to the type of treatment they received, the hygienist performing the measurements and the scaling was also blinded to group allocation.

Another strength is the prolonged follow up that allowed use to assess the effect of the toothpastes before and long after a scaling intervention.

Moreover, the calibration and standardization of the hygiene instructions for all patients was also a strength. All patients used exactly the same oral hygiene kit, following the same oral hygiene instructions.

### Example 8

### Composition of CB Before and After Treatment

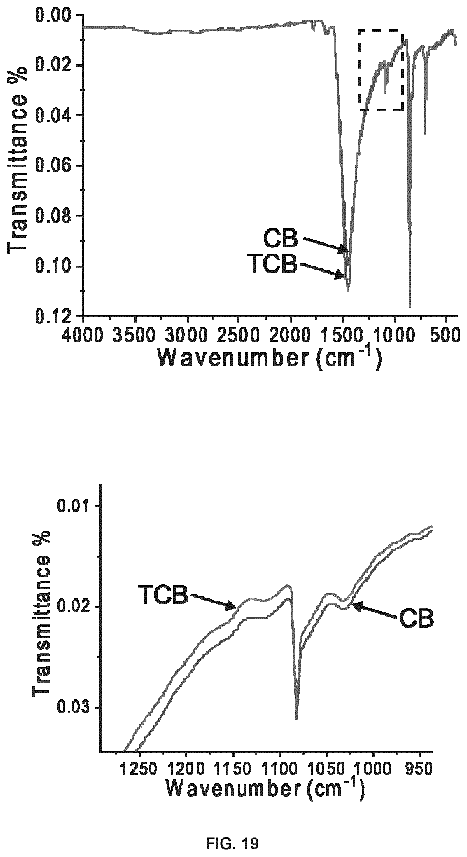

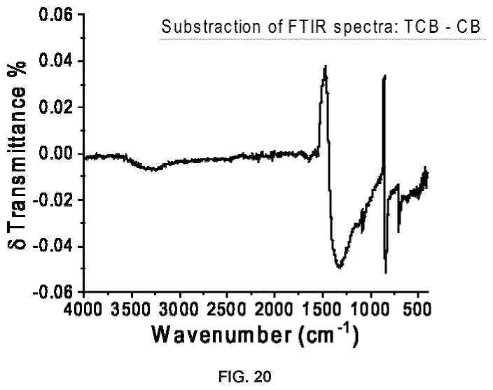

Now referring to FIGS. 19 and 20. Treatment of the CB with the process detailed in Example 1, above, affects the intensity of the transmittance peaks related to chitin at 1030 and 1150, and 1125 cm⁻¹. Treatment of the CB decreased the full width at half maximum of the carbonate peak at 1490 cm⁻¹ indicating an increase in crystallinity

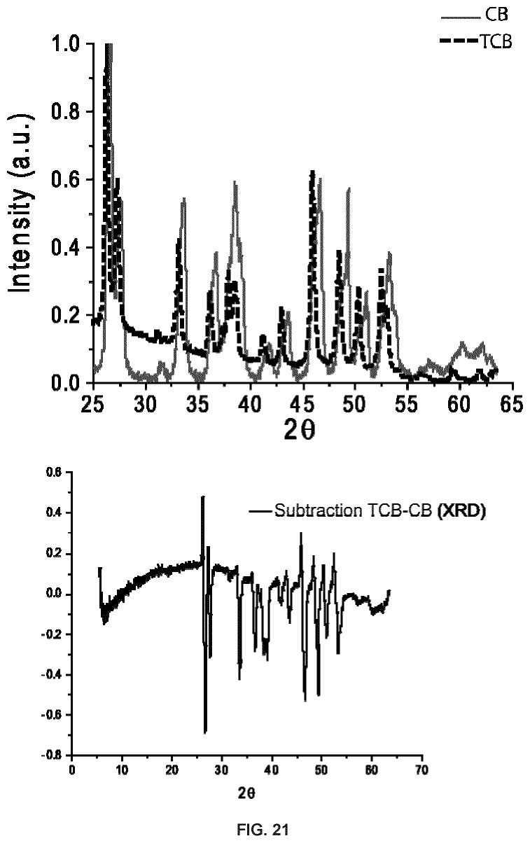

Now referring to FIG. 21. Treatment of the CB with the process detailed in Example 1, above, XRD reveals that treatment increases the cell lattice of CB. This is confirmed by the fact that the diffraction peaks of aragonite in CB shift to lower angles after treatment. XRD also reveals that treatment increases the crystallinity of CB. This is confirmed by the fact that the diffraction peaks become sharper after treatment with the process.

### Example 9

### Effect of TCB Calcium Phosphate Precipitation

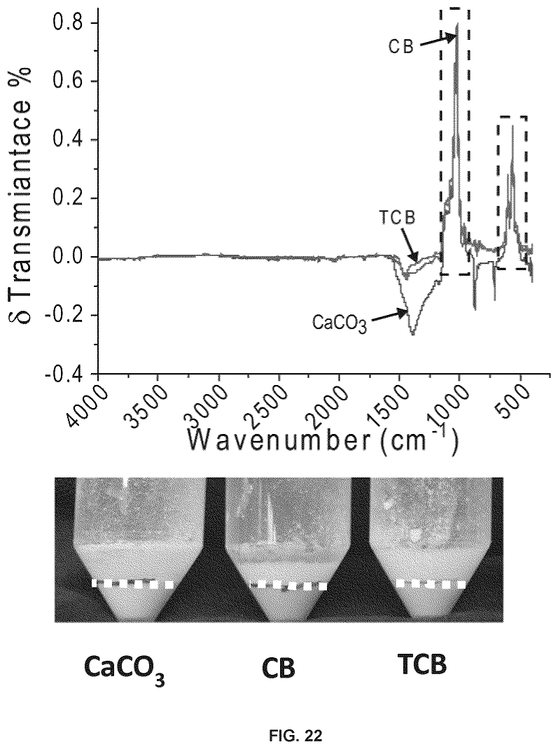

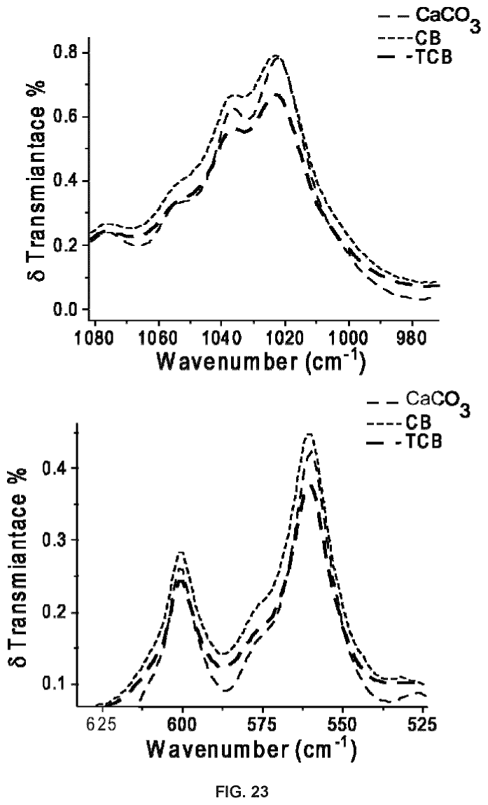

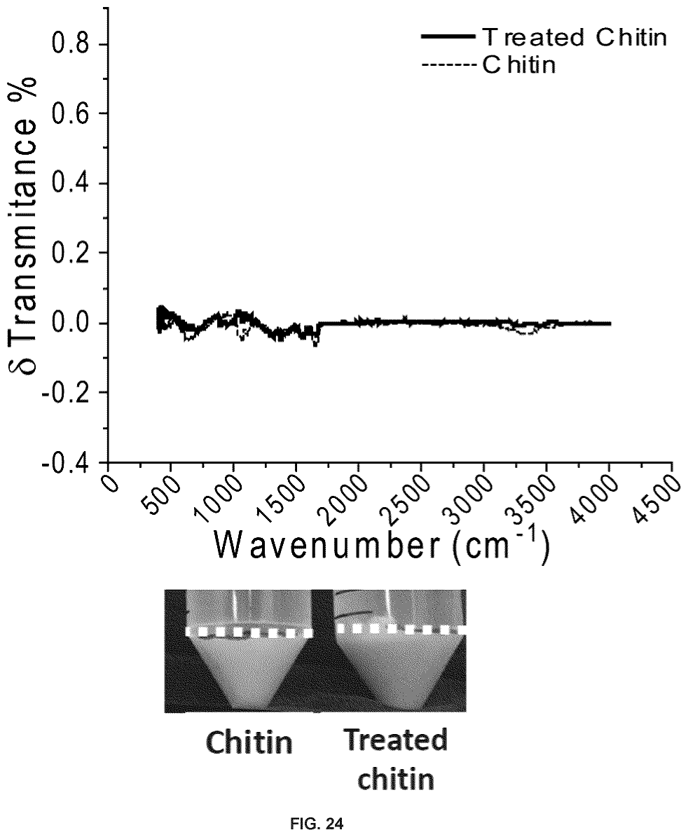

TCB samples were incubated in super-saturated solution of calcium phosphate. CB and synthetic calcium carbonate (calcite) were used as controls. Briefly, 0.5 grams of each powder (CB, TCB, chitin, treated chitin and CaCO₃ were incubated at 37° C. for 1, 3, 7 or 10 days in a supersaturated solutions of 15 mM CaCl₂) and 15 mM NaHPO₄ at a pH 5.6. After incubation the suspensions were centrifuged at 10,000 rpm for 15 minutes and the precipitates were collected, dried and kept for analysis. Samples were analyzed by FTIR and XRD before and after incubation. All experiments were performed in triplicates. Now referring to FIGS. 22 and 23, FTIR analysis of synthetic Calcium carbonate, Cuttlebone, and treated cuttlebone shows the difference in transmittance spectra between baseline measurements and measurements after incubation in supersaturated solutions of calcium phosphate. TCB shows lower signal for phosphate groups, and higher signal for carbonate groups indicating that it has an inhibitory effect on calcium phosphate precipitation.

FTIR analysis of chitin and treated chitin, shows the difference in transmittance spectra between baseline measurements and measurements after incubation in supersaturated solutions of calcium phosphate. Both materials show almost no traces for phosphate groups indicating a potential effect as crystal growth inhibitors. The Chitin shows more changes in the spectra than treated chitin, indicating that the treated chitin might be slightly less soluble in water.

### Example 10

### Effect of TCB on Dental Calculus

9 samples of human dental calculus were mounted into resin blocks mirror polished and sonicated in distilled water to remove debris. This provided standardized flat surfaces of dental calculus. The mounted calculus specimens were exposed to a slurry of TCB by mixing the powders with distilled water at a 1:1 (w:w) for 1 h, and changes in the calculus surface were assessed by XRD, SEM, EDX, and profilometry. The experiments were repeated with slurries of CB and synthetic calcium carbonate, as well as with distilled water as controls. The embedded calculus samples were immersed in the slurry for 1 hour. Then collected and rinsed with ddH₂O and dried. The effect of each slurries on the calculus removal was determined by grazing angle-ray diffraction and SEM.

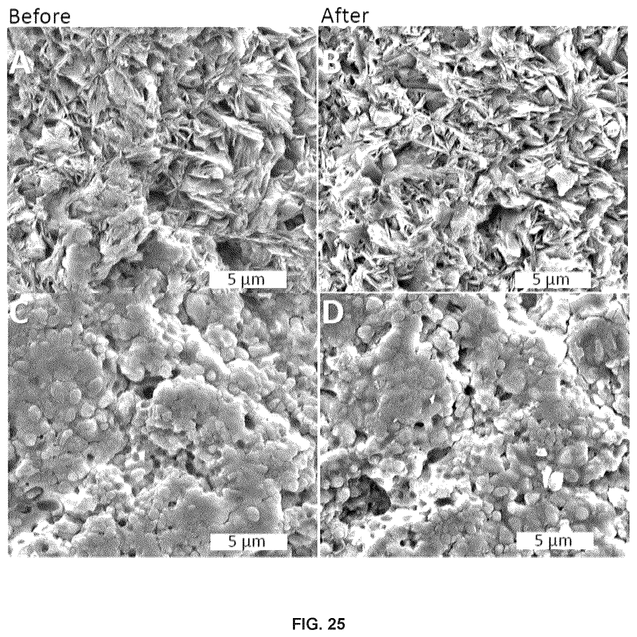

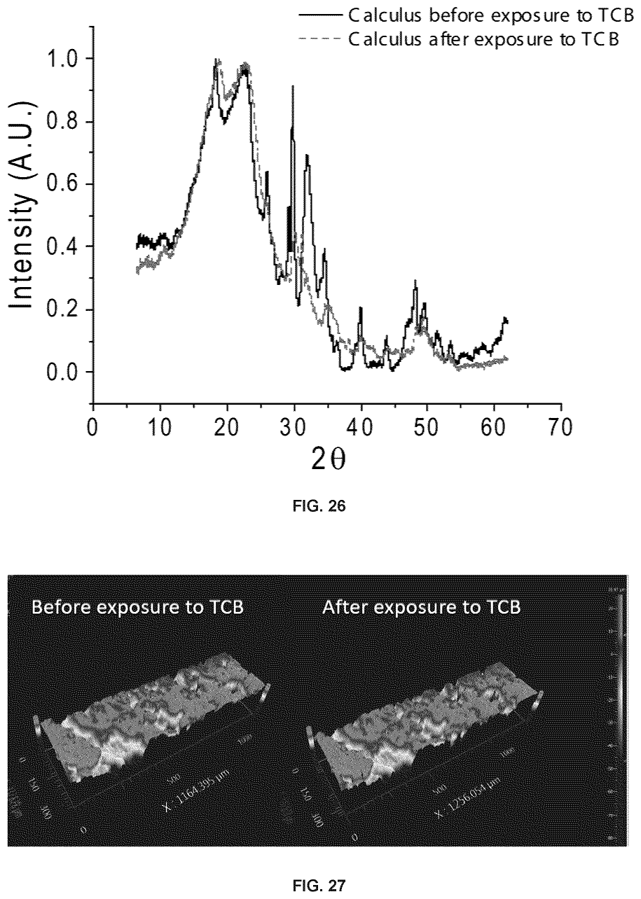

Now referring to FIGS. 25A-D and 26, upon exposure to cuttlebone powder, dental calculus roughness increased through partial dissolution of its nanocrystals. 25A-D show SEM micrographs reveals partial dissolution of nanocrystals on the surface of calculus after exposure to cuttlebone (TCB) slurry (Scalebar=5 microns). FIG. 26 shows grazing angle XRD diffractogram of dental calculus powder before and after exposure to cuttlebone slurry which reveals a decrease in crystallinity apparent from the increase of the relative width of the diffraction peaks.

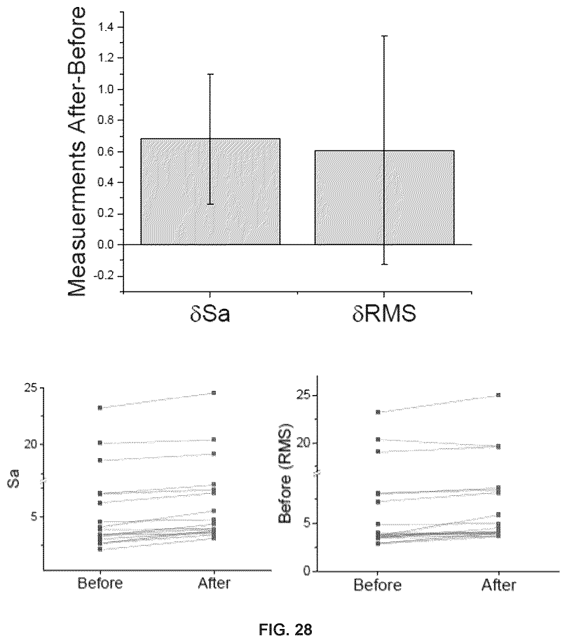

Now referring to FIGS. 27 and 28, it is shown that exposure to TCB increases the surface roughness of dental calculus. FIG. 27 is a profilometry analysis which reveals an increase in calculus surface roughness after exposure to TCB slurry. FIG. 28 shows increase in the trends before and after treatment.

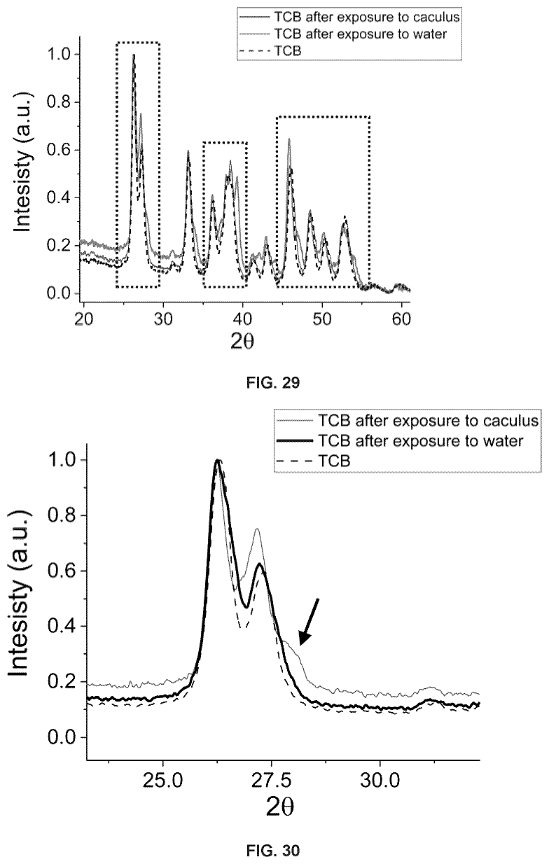

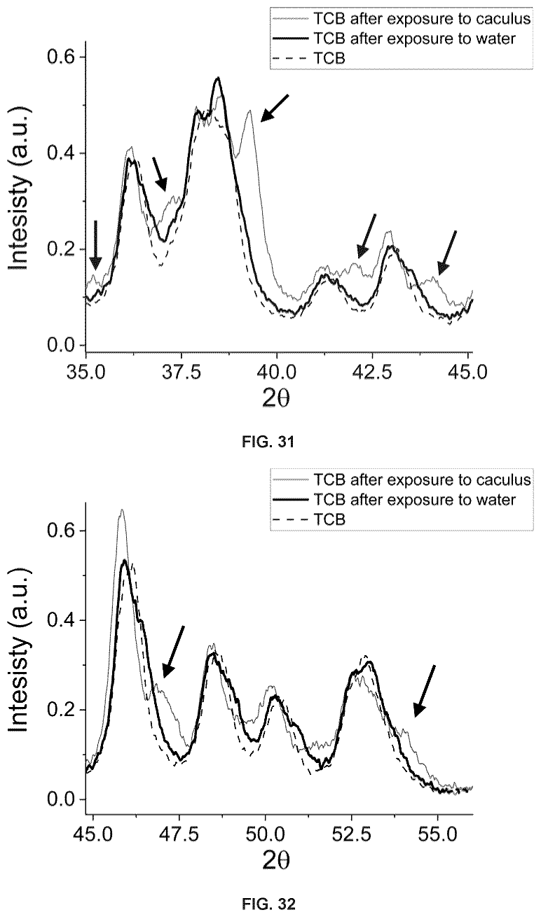

Next, the composition of TCB was assessed after exposure to dental calculus. Referring now to FIGS. 29 to 32, it is shown that there is a change in TCB composition upon exposure to calculus, as XRD reveals an overall shift of the cuttlebone aragonite peaks towards lower angles after exposure to calculus. New diffraction peaks appear at 46.9, 54.1, 27.9, 37.5, 35.1, 39.36, 42, 44.1. FIG. 29 shows the XRD over the whole range, which FIGS. 30-32 are focus on the inset stippled boxes of FIG. 29.

TABLE 5
Values control versus treatment

| Cell lattice | | |
|---|---|---|
| parameter | Control | Treated |
| a | 4.94 | 4.962 |
| b | 7.94 | 7.968 |
| C | 5.72 | 5.743 |
| V | 224.36 | 227.11 |

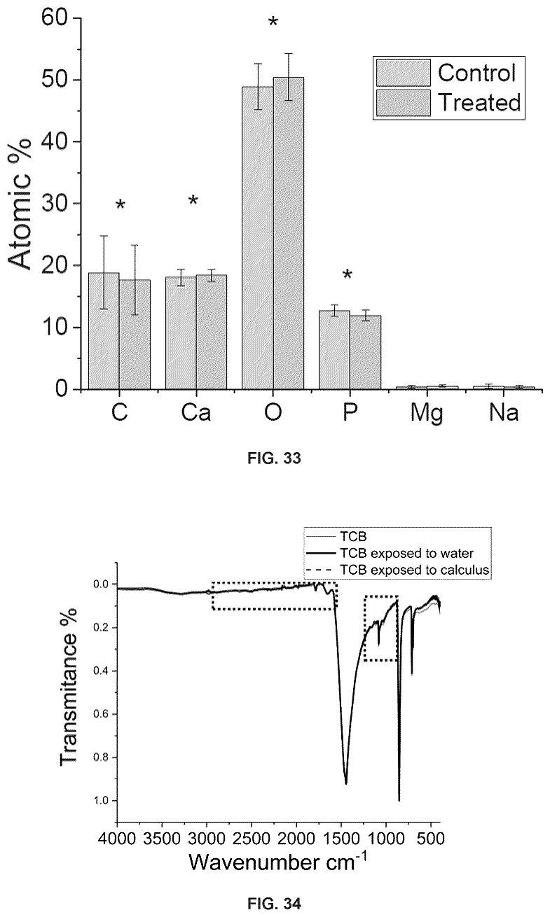

Next, the elemental composition of dental calculus was assessed before and after exposure to the TCB slurry. Now referring to FIG. 33, which is an EDX elemental analysis of dental calculus before and after exposure to TCB slurry. This shows that exposure to TCB decreased phosphate and carbon content while increasing calcium and oxygen relative content. TCB also increased magnesium content and decreased sodium content in calculus.

### Example 11

### Effect of Dental Calculus on TCB

As for Example 10, 9 samples of human dental calculus were mounted into resin blocks mirror polished and sonicated in distilled water to remove debris. This allowed having standardized flat surfaces of dental calculus. The mounted calculus specimens were exposed to slurry of TCB in distilled water (1:1 w/w) for 1 h, and changes in the TCB and calculus were assessed by XRD, SEM, EDX, and FTIR. Changes in the slurries pH were measured with a pH-meter. The experiments were repeated with slurries of CB and synthetic calcium carbonate, as well as with distilled water as controls.

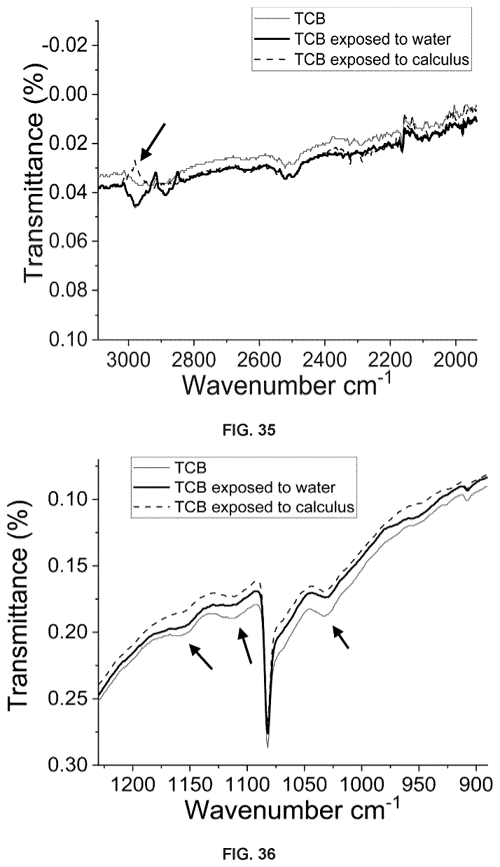

Now referring to FIGS. 34 to 36, which show FTIR spectra for TCB upon exposure to dental calculus or distilled water. FIG. 34 shows the whole spectra, which FIGS. 35 and 26 are focused on the inset stippled boxes of FIG. 34. The results show that TCB slurry releases traces of chitin upon exposure to dental calculus. Indeed, the FTIR analysis reveal changes in the cuttlebone absorbance spectra at the 1025,1120,1160 and 3000 cm⁻¹, which indicates that CB powder loses chitin upon exposure to calculus.

TABLE 6
pH of TCB slurry

| Sample | pH |
|---|---|
| dd-H₂O | 5.56 ± 0.011 |
| TCB-dd-H₂O | 7.92 ± 0.047 |
| Calculus-TCB-dd-H₂O | 8.08 ± 0.02 |

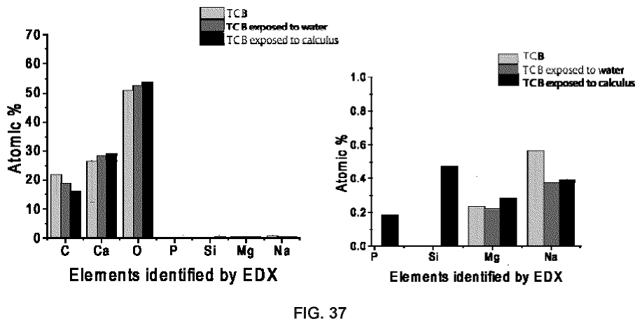

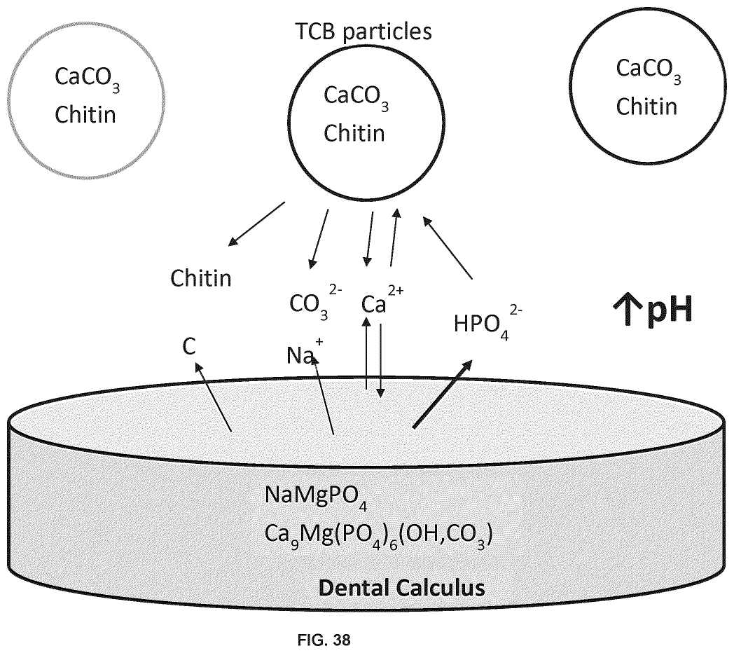

Now referring to FIG. 37, the presented EDX elemental analysis of TCB before and after exposure to dental calculus shows that exposure to TCB decreased carbon content while increasing calcium, oxygen and phosphate relative content. TCB also increased magnesium content and decreased sodium content in TCB.

In conclusion, TCB appears to inhibit the precipitation of calcium phosphate, possibly due to its chitin content. Exposing dental calculus to TCB slurry results in changes in the chemical, elemental and crystallographic composition of TCB. Changes in the chemical, elemental and crystallographic composition of dental calculus. Also, there is an increased surface roughness of dental calculus.

### Example 12

### Comparison of TCB and TCB Particles of 60-61 Microns

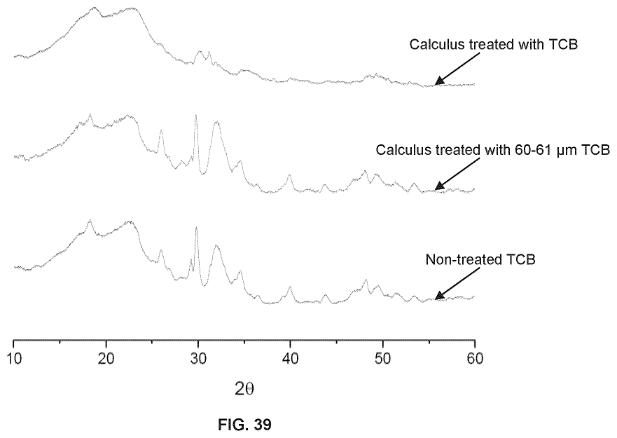

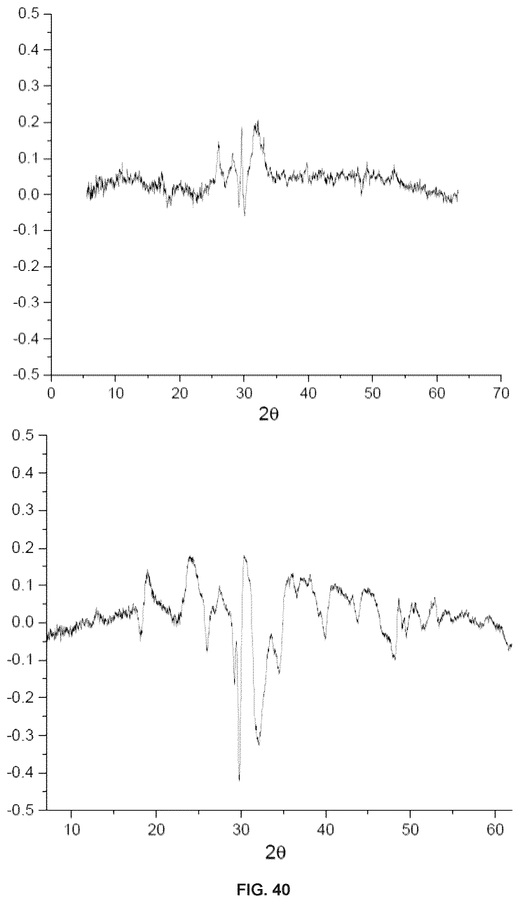

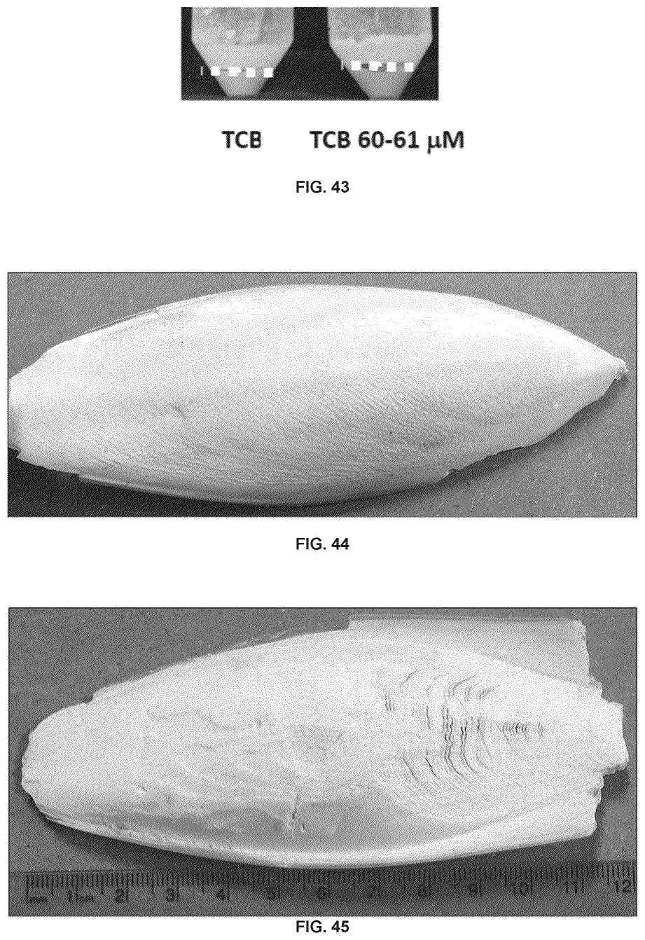

As for Example 10, 9 samples of human dental calculus were mounted into resin blocks mirror polished and sonicated in distilled water to remove debris. This allowed having standardized flat surfaces of dental calculus. The mounted calculus specimens were exposed to slurries of TCB or TCB particles of 60-61 μm in distilled water (1:1 w:w) for 1 h, and changes in the TCB or TCB 60-61 μm and calculus were assessed by XRD and FTIR. The effect of slurries on the calculus was determined by X-ray diffraction and calculus was analyzed by XRD and FTIR before and after treatment with the TCB and TCB 60-61 μM TCB. Now referring to FIGS. 39 to 43. FIG. 39 shows the XRD of calculus sample after reaction with the TCB and 60-61 μm TCB, showing that the spectra of TCB is different than that of 60-61 μm TCB and untreated control. FIG. 40 shows the subtraction of the spectra, where the top graph shows the subtraction of the 60-61 μm TCB and the untreated control, and the bottom graph shows that subtraction of the TCB from the untreated control. As can be seen from FIGS. 39-40, the reaction between the TCB, and calculus is greater than the reaction with the 60-61 μm TCB, which indicates that the reaction leads to greater dissolution and decrease of the calculus main crystalline peaks.

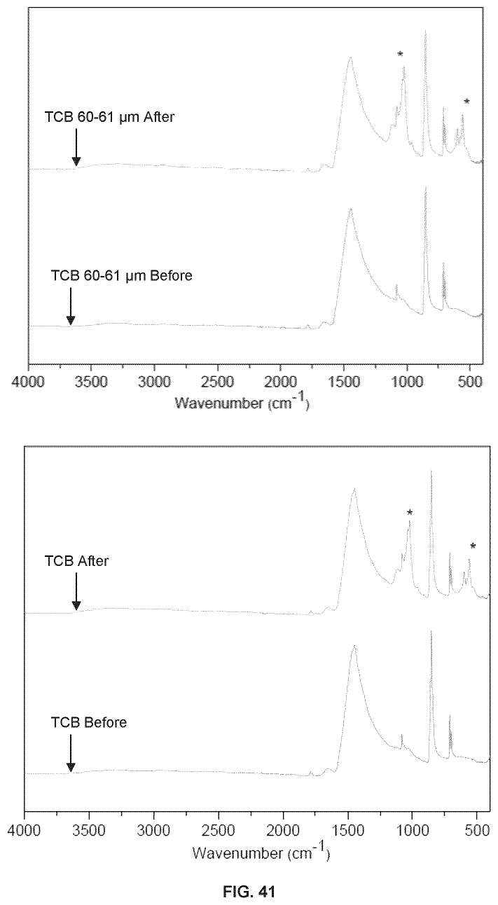

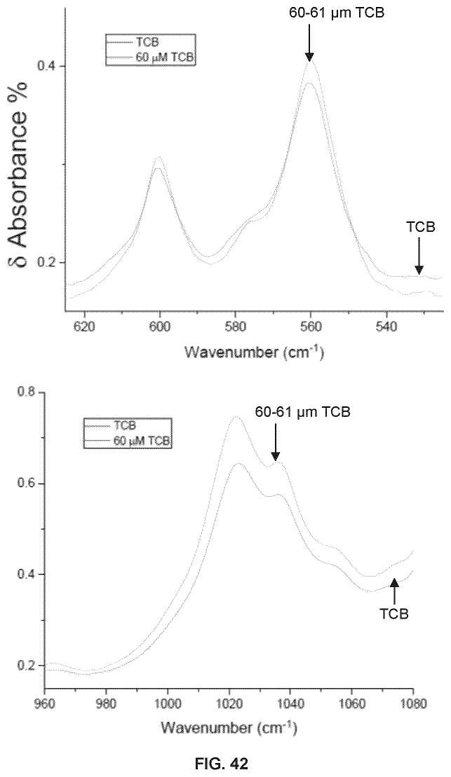

Next, the effect of TCB and 60-61 μm TCB on calcium phosphate precipitation was tested. TCB and 60-61 μm TCB samples were incubated in a super-saturated solution of calcium phosphate. Samples were analyzed by FTIR and XRD before and after incubation. All experiments were performed in triplicates. Now referring to FIG. 41, the FTIR spectra of the TCB and 60-61 μm TCB before and after reacting with super-saturated solution of calcium phosphate is shown. FIG. 42 shows a zoomed view of the FTIR spectra between the indicated wavenumber (identified by the * in FIG. 41). The results show the difference in the absorbance spectra between baseline measurements and measurements after incubation in supersaturated solutions of calcium phosphate. TCB shows lower signal for phosphate groups than 60-61 μm TCB, indicating that it has a stronger inhibitory effect on calcium phosphate precipitation.

### Example 13

### Process for Separation of Cuttlefish Bone Ventral Chamber and Dorsal Shield

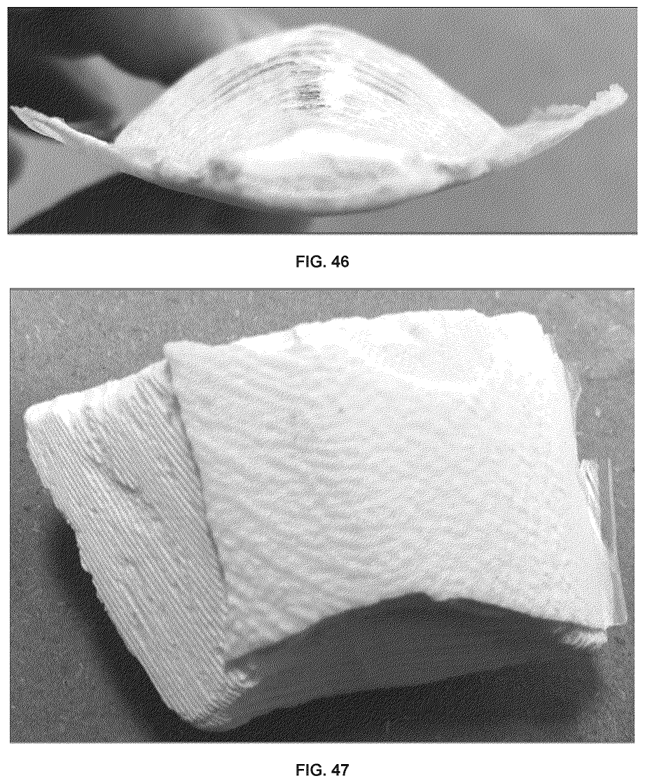

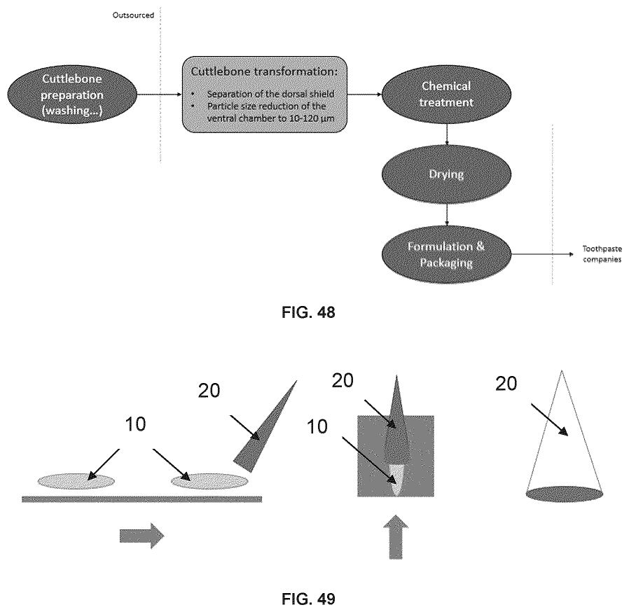

The present invention is prepared from cuttlefish bones (or cuttlebones) as a raw material. Cuttlebone is the bone of a cuttlefish, an animal similar to an octopus. Their bones are mostly made of calcium carbonate (CaCO₃) in the form of aragonite compared to other structures of calcium carbonate such as calcite that can be found in limestone. Cuttlebones are composed of two distinct sections having different mechanical properties: the dorsal shield and the lamellar matrix or ventral chamber. The dorsal shield is visible on FIG. 44. The dorsal shield is a rigid and dense layer while the ventral chamber is more brittle and crumbles like chalk. The ventral chamber (chalk) corresponds to what can be seen on FIG. 45. FIGS. 46-47 represent a frontal view of a cuttlebone (dorsal shield on the bottom) and a damaged cuttlebone piece showing the dorsal shield on top and the ventral chamber (lamellar matrix) underneath, respectively. FIG. 48 represents a flowchart of the different processing steps leading to the preparation of TCB according to the present invention.

### Cuttlebone Physical Characteristics

### Raw Material Density

The cuttlebones are made mainly composed of aragonite (CaCO₃). The two sections of the cuttlebone have different densities (0.4 g/cm³ for the ventral chamber vs 2.4 g/cm³ for the dorsal shield). This density difference relies on the structure of the ventral chamber, which has a lot more air pockets than the denser dorsal shield. This density difference between both sections of the bone decreases greatly when the cuttlebone in ground into small particles. The smaller the bone particles get, the smaller the density difference between the two bone components gets. Density values are: Bulk density of the cuttlebones: 0.17 g/cm³. Ventral chamber density (whole): 0.4 g/cm³. Dorsal shield density (whole): 2.4 g/cm³. Ventral chamber density (mean density for the desired particle size distribution): 2.42 g/cm³. Dorsal shield density (mean density for the desired particle size distribution): 2.59 g/cm³.

### Chemical Composition

The following table details the composition of both cuttlebone sections. From this table, it can be concluded that both the ventral chamber and the dorsal shield have very similar chemical compositions.

TABLE 7
Cuttlebone chemical composition

| | Ventral chamber | Dorsal shield |
|---|---|---|
| | (% w/w) | (% w/w) |
| CaCO₃ | 96.70 | 97.40 |
| Na₂O | 1.29 | 0.75 |
| Cl | 0.76 | 0.44 |
| SrO | 0.33 | 0.27 |
| SO₃ | 0.29 | 0.38 |
| MgO | 0.26 | 0.15 |
| P₂O₅ | 0.18 | 0.07 |
| K₂O | 0.08 | 0.04 |
| SiO₂ | 0.04 | 0.03 |
| Fe₂O₃ | 0.01 | 0.28 |

The main difficulty of the process described herein is the unusual physical properties of the cuttlebones and the irregularity of their shape from one cuttlebone to another. It was also noted that when the cuttlebones are put in water, whole bones do not get oriented on one particular side, which may have been helpful to make sure all cuttlebones are on the same side before a treatment to remove the ventral chamber for example.

As mentioned above, the density difference between both bone sections is greatly reduced when the bones are grinded. Furthermore, the chemical composition of both sections is very similar. These two characteristics make the separation of the dorsal shield and the ventral chamber more challenging. The separation process must be sanitary (food grade), since the product will be used in the formulation of toothpaste for human use. Cleanability must also be considered for the technology selected.

The following hypotheses were made regarding cuttlebones: the dorsal shield corresponds to about 50% w/w of the cuttlebone. This hypothesis is used as a worst-case scenario since the percentage of dorsal shield varies between 35 and 50%. Cuttlebones are not sensitive to heat. Cuttlebones are not flammable and cuttlebone powder is not combustible since it is made of calcium carbonate at more than 95%. Cuttlebones are not hygroscopic. Cuttlebone separation can be done continuously or in batches since the next process step (chemical treatment) will be performed in batches. The maximum percentage of dorsal shield in the finished product powder is 10%.

Water jets may provide enough impact on the ventral chamber to remove the ventral chamber while the dorsal shield would stay intact. The first tests were performed with a high-pressure water jet, a water jet cutter. The pressures tested, around 94 000 psi (about 648.1 MPa), were too high and damaged the dorsal shield. Second tests were conducted with an industrial water pressure washer at approximately 3000 psi (about 20.7 Mpa) with a water jet of 4 inches and the pressure was still too high and damaged the dorsal shield. Next, tests were performed with a home water pressure washer and lowered the pressure to approximately 800 psi (about 55 Mpa) to obtain favorable results. At this pressure, for the nozzle on the equipment and the distance used between the nozzle and the cuttlebone, it was possible to remove the ventral chamber and keep the dorsal shield intact.

Now referring to FIG. 49 which illustrates cuttlebone (10) being contacted with water jet (20), when under movement counter-currently to the water jet. The process parameters include a flat nozzle with an elliptical pulverization shape aligned with the middle of the width of the cuttlebones to ensure that there is a greater impact in the middle of the cuttlebone, where the ventral chamber is thicker. A narrow nozzle pulverization, between 25 and 40 degrees, oriented with a slight angle counter-current of the cuttlebones, meaning that the nozzles are not be perpendicular with the cuttlebones to avoid crushing the cuttlebones and promote the removal of the ventral chamber. There is a distance of 2 to 4 inches (about 5 to 10 cm) between the nozzle and the cuttlebone to ensure that the pulverization shape is maintained. The cuttlebone would be moved at an estimated speed of 100 feet per minute (about 0.5 m/s or 30 m/min).

Other parameters to consider could include water recycling and a possible concentration step, if required, before the next process step (chemical treatment of the particles) in order to obtain the appropriate particle concentration in the reactor for the chemical treatment.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. An oral care composition comprising:
a cuttlefish bone powder, comprising particles having more than 95% (w/w) calcium carbonate content, a specific surface area of at least 5 m²/g, a mechanical hardness about 4.75 to about 7 GPa, and at least 20% of said particles of said powder have a particle size of from about 50 microns to about 70 microns and a mean of about 60 microns, and
a suitable carrier.

2. The oral care composition of claim 1, wherein said calcium carbonate content is from about 95% to about 99.9%.

3. The oral care composition of claim 1, wherein said particles have a specific surface area of from about 5 m²/g to about 8 m²/g.

4. The oral care composition of claim 1, wherein said particles have a mechanical hardness of about 4.75 GPa, or about 6.87 GPa.

5. (canceled)

6. The oral care composition of claim 1, wherein said particles have a zeta potential of about −11.4 to about −12.6 mV, or about −12 mV.

7. (canceled)

8. The oral care composition of claim 1, wherein said cuttlefish bone powder is from about 0.100% to about 20% (w/w) of the composition.

9. The oral care composition of claim 1, wherein at least 60% of said particles of said powder have a particle size of from about 10 microns to about 70 microns, or wherein at feast 55% of said particles of said powder have a particle size of from about 10 microns to about 60 microns, or wherein at least 50% of said particles of said powder have a particle size of from about 10 microns to about 50 microns, or wherein at least 40% of said particles of said powder have a particle size of from about 10 microns to about 45 microns, or wherein at least 35% of said particles of said powder have a particle size of from about 10 microns to about 40 microns, or wherein at least 30% of said particles of said powder have a particle size of from about 10 microns to about 35 microns, or wherein at least 25% of said particles of said powder have a particle size of from about 10 microns to about 30 microns, or wherein at least 20% of said particles of said powder have a particle size of from about 10 microns to about 26 microns, or wherein at least 15% of said particles of said powder have a particle size of from about 10 microns to about 23 microns, or wherein at least 12% of said panicles of said powder have a particle size of from about 10 microns to about 20 microns, or wherein at least 3% of said particles of said powder have a particle size of from about 10 microns to about 17 microns, or wherein at least 6% of said particles of said powder have a particle size of from about 10 microns to about 15 microns, or wherein at least 50% of said particles of said powder have a particle size of from about 10 microns to about 175 microns, or combinations thereof.

10.-21. (canceled)

22. The oral composition of claim 1, further comprising at least one of:
an abrasive;
a thickening agent;
a humectant;
an emulsifier;
a surfactant;
a pH regulator;
a solvent;
an antimicrobial agent; and
a preservative.

23. The oral care composition of claim 22, wherein said abrasive is a colloidal calcium, a colloidal silica, a hydrated silica, a sodium bicarbonate (NaHCO₃), aluminum hydroxide (Al(OH)₃), calcium carbonate (CaCO₃), a calcium hydrogen phosphate (CaHPO₄.2H₂O), an anhydrous calcium hydrogen phosphate, a silica, a zeolites, and hydroxyapatite (Ca₅(PO₄)₃OH), or a combination thereof.

24. The oral care composition of claim 22, wherein said abrasive is from about 0.100% to about 0.325% (w/w) of the composition, and wherein said colloidal silica is from about 0.100% to about 0.275% (w/w), or from about about 0.02% to about 0.08% (w/w) of the composition.

25.-27. (canceled)

28. The oral care composition of claim 22, wherein said thickening agent is a natural gum obtained from seaweeds; a natural gum obtained from non-marine botanical resource, a natural gum produced by bacterial fermentation, a starch, a pectin, a carboxymethyl cellulose, a hydroxypropyl cellulose, a methyl cellulose, a gelatin or a combination thereof.

29. The oral care composition of claim 28, wherein said natural gums obtained from seaweeds is chosen from agar (E406), alginic acid (E400), Sodium alginate (E401), potassium alginate, ammonium alginate, calcium alginate, carrageenan (E407), or a combination thereof;
wherein said natural sum obtained from non-marine botanical resource is chosen from acacia gum, gum arable (E414), gum ghatti, gum tragacanth (E413), karaya gum (E416), guar gum (E412), locust bean gum (E410), beta-glucan, chicle gum, dammar gum, Glucomannan (E425), mastic gum, psyllium seed husks, spruce gum, tare gum (E417), or a combination thereof;
wherein said natural gum produced by bacterial fermentation is chosen from gellan gum (E418), Xanthan gum (E415), or a combination thereof.

30.-31. (canceled)

32. The oral care composition of claim 27, wherein said thickening agent is from about 2% to about 66% (w/w), or about 2.2% (w/w) of the composition.

33.-34. (canceled)

35. The oral care composition of claim 22, wherein said humectant is propylene glycol, hexylene glycol, butylene glycol, glyceryl triacetate, neoagarobiose, a sugar polyol, a polymeric polyol, quillaia, lactic acid, urea, glycerin, aloe vera gel, MP Diol, an alpha hydroxy acid, and honey.

36.-39. (canceled)

40. The oral care composition of wherein said humectant is from about 2% to about 5% (w/w) of the composition.

41.-44. (canceled)

45. The oral care composition of claim 22, wherein said emulsifier is from about 4% to about 10% (w/w) of the composition.

46. (canceled)

47. The oral composition of claim 22, wherein said surfactant is chosen from sodium lauryl sulfate, ammonium lauryl sulfate, sodium N-lauryl sarcosinate, sodium lauryl sulfoacetate, or a combination thereof.

48. The oral composition of claim 22, wherein said surfactant is from about 1% to about 3% (w/w) of the composition.

49.-55. (canceled)

56. The oral composition of claim 22, wherein said solvent is chosen from water, ethanol, isopropanol, sorbitol and glycerin.

57. The oral composition of claim 22, wherein said solvent is from about 60% to about 99% (w/w) of said composition.

58.-62. (canceled)

65. A method of cleaning an oral cavity comprising applying the oral composition of claim 1 to an oral cavity.

66. A method of preventing formation of, or of removing calculus in an oral cavity comprising applying the oral composition of claim 1 to an oral cavity.

67.-71. (canceled)
